(19) United States
(12) Patent Application Publication
(10) Pub. No.: US 2009/0088466 A1
(43) Pub. Date: Apr. 2, 2009

(54) OPIOID RECEPTOR LIGANDS

(75) Inventors: Laura M. Bohn, Upper Arlington, OH (US); Thomas E. Prisinzano, Coralville, IA (US)

Correspondence Address: VIKSNINS HARRIS & PADYS PLLP, P.O. BOX 111098, ST. PAUL, MN, 55111-1098, US

(73) Assignee: UNIVERSITY OF IOWA RESEARCH FOUNDATION, Iowa City, IA (US)

(21) Appl. No.: 11/917,314

(22) PCT Filed: Jun. 16, 2006
(86) PCT No.: PCT/US2006/023528

§ 371 Date: Oct. 20, 2008

Related U.S. Application Data
(60) Provisional application No. 60/692,010, filed on Jun. 17, 2005.

(51) Int. Cl. A61K 31/366; C07D 405/04; C07K 1/113

(52) U.S. Cl. 514/455; 549/280; 530/410

(57) ABSTRACT

The invention provides novel compounds of formula (I), (II), (III), and (IV). The invention also provides pharmaceutical compositions comprising such compounds as well as methods for treating diseases associated with opioid receptor function by administering such compounds to an animal in need of treatment. The invention also provides therapeutic methods for the use of compounds of formula (V), as well as methods for treating diseases by administering such compounds.

### RELATED APPLICATION

This application claims priority to U.S. Provisional Application No. 60/692,010 filed 17 Jun. 2005.

### BACKGROUND OF THE INVENTION

The opium poppy, Papaver somniferum, has been used for centuries for the relief of pain and to induce sleep (Casy, A. F.; Parfitt, R. T. Opioid analgesics: chemistry and receptors; Plenum Press: New York, 1986; xv, 518). Among the most important constituents in opium are the alkaloids morphine and codeine. Many of the agonists and antagonists derived from these alkaloids are essential for the practice of modern medicine. While many potent agonists are effective analgesics, they have undesirable side effects, such as tolerance, dependence, and respiratory depression. (Stein, C.; Schafer, M.; Machelska, H. Nat. Med. 2003, 9, 1003-1008).

Endogenous opioid peptides are known and are involved in the mediation or modulation of a variety of mammalian physiological processes, many of which are mimicked by opiates or other non-endogenous opioid ligands. Some of the processes that have been suggested include analgesia, tolerance and dependence, appetite, renal function, gastrointestinal motility, gastric secretion, respiratory depression, learning and memory, mental illness, epileptic seizures and other neurological disorders and cardiovascular responses.

Intensive research of the last two decades has given us a better understanding of opioid receptor structure, distribution, and pharmacology (Waldhoer, M.; Bartlett, S. E.; Whistler, J. L. Annu. Rev. Biochem. 2004, 73, 953-990). Three types of opioid receptors known as mu (μ), delta, (δ), and kappa (κ) and receptor subtypes have been identified, and the mRNA encoding these receptors has been isolated. There is substantial pharmacological evidence for subtypes of each (Reisine, T. Neurotransmitter Receptors V: Opiate Receptors. Neuropharmacology 1995, 34, 463-472) It has become clear that each receptor mediates unique pharmacological responses and is differentially distributed in the central nervous system (Goldstein, A.; Naidu, A., Mol. Pharmacol. 1989, 36, 265-272; and Mansour, A.; Fox, C. A.; Akil, H.; Watson, S. J., Trends Neurosci. 1995, 18, 22-29).

The endogenous ligands for the opioid receptors are neuropeptides (Casy, A. F.; Parfitt, R. T. Opioid analgesics: chemistry and receptors; Plenum Press: New York, 1986; xv, 518). To date, three families of endogenous opioid peptides have been identified. They are classified, β-endorphins, enlcephalins, and dynorphins (Gutstein, H.; Alcil, H. Opioid Analgesics. Goodman & Gilman's The Pharmacological Basis of Therapeutics; 10th ed.; McGraw-Hill: New York, 2001; pp 569-619; and Eguchi, M., Med. Res. Rev. 2004, 24, 182-212). Although most of these endogenous opioids have little selectivity for opioid receptors, it is generally accepted that β-endorphins, enlcephalins, and dynorphins display greater affinity for μ, δ and κ receptors respectively.

There are several structural classes of nonpeptidic opioid receptor ligands (Eguchi, M., Med. Res. Rev. 2004, 24, 182-212; Kaczor, A.; Matosiuk, D., Curr. Med. Chem. 2002, 9, 1567-1589; and Kaczor, A.; and Matosiuk, D., Curr. Med. Chem., 2002, 9, 1591-1603). The oldest class of compounds are those derived from morphine (2) (FIG. 1). Examples of other structural classes include fentanyl (3), cyclazocine (4), SNC 80 (5), U50,488H (6), and 3FLB (7) (see FIG. 1). The common structural motif in all of these ligands is the presence of a basic amino group.

Salvinorin A is a unique opioid receptor ligand (1, FIG. 1). It bears little structural similarity to other structural classes of nonpeptidic opioid receptor ligands such as morphine, fentanyl, cyclazocine, SNC 80, U50,488H, and 3FLB, which all possess a basic amino group. Until recently it has been assumed that the presence of a positively charged nitrogen atom in opioid compounds represented an absolute requirement for their interaction with opioid receptors (Rees, D. C.; Hunter, J. C. Comprehensive Medicinal Chemistry; Pergammon: New York, 1990; pp 805-846). The general assumption was that this cationic amino charge on the opioid ligand would interact with the side chain carboxyl group of an aspartate residue located in TM III of the opioid receptor (Eguchi, M., Med. Res. Rev. 2004, 24, 182-212; Surratt, C.; Johnson, P.; Moriwaki, A.; Seidleck, B.; Blaschak, C. et al. J. Biol. Chem. 1994, 269, 20548-20553; and Lu, Y.; Weltrowska, G.; Lemieux, C.; and Chung, N. N.; Schiller, P. W., Bioorg. Med. Chem. Lett., 2001, 11, 323-325). Given the structure and potency of salvinorin A (1), this interaction is unlikely.

Salvinorin A, originally isolated from the leaves of Salvia divinorum, was found to be very selective for κ receptors over μ and δ opioid receptors, as well as over a battery of other receptors. This was the first report of a nonnitrogenous κ opioid receptor agonist (Ortega, A.; Blount, J. F.; Manchand, P. S. Salvinorin, J. Chem. Soc. Perkin Trans. 1, 1982, 2505-2508; Valdes III, L. J.; Butler, W. M.; Hatfield, G. M.; Paul, A. G.; Koreeda, M. Divinorin A, J. Org. Chem. 1984, 49, 4716-4720; and Roth, B. L.; Baner, K.; Westkaemper, R.; Siebert, D.; Rice, K. C. et al., Proc. Natl. Acad. Sci. USA 2002, 99, 11934-11939). The pharmacology of salvinorin A appears to be different than other K agonists (Wang, Y.; Tang, K.; Inan, S.; Siebert, D. J.; Holzgrabe, U; Lee, D. Y. W.; Huang, P.; Li, J. G.; Cowan, A.; and Liu-Chen, L.-Y., J. Pharmacol. Exp. Ther. 2004, 312, 220-230).

GPCR internalization has been a particularly stimulating topic among opioid receptor research and this means of regulation has been associated with conditions as wide-ranging as opioid analgesic tolerance to opioid addiction (Alvarez, V., Arttamangkul, S. & Williams, J. T., 2001, Neuron 32, 761-3; Connor, M., Osborne, P. B. & Christie, M. J., 2004, Br J Pharmacol 143, 685-96; Gainetdinov, R. R., Premont, R. T., Bohn, L. M., Lefkowitz, R. J. & Caron, M. G., 2004, Annu Rev Neurosci 27, 107-44; Bohn, L. M., Gainetdinov, R. R. & Caron, M. G., 2004, Neuromolecular Med 5, 41-50; and Raehal, K. M. & Bohn, L. M., 2005, Aaps J 7, E587-91).

As a GPCR, the opioid receptor is subject to agonist-induced, GPCR kinase (GRK)-mediated phosphorylation, subsequent β-arrestin protein binding, the assembly of clathrin coated vesicles and vesicular internalization (Shenoy, S. K. & Lefkowitz, R. J., 2003, Biochem J, 375, 503-15; and Pierce, K. L. & Lefkowitz, R. J., 2001, Nat Rev Neurosci, 2, 727-33). This is a general paradigm for GPCR internalization, yet the μ opioid receptors (μOR) have proven to be differentially regulated by agonist occupancy. For example, while both morphine and etorphine are agonists at the μOR and can promote receptor desensitization and analgesic tolerance, morphine appears to be much less effective in promoting receptor phosphorylation, β-arrestin recruitment, and μOR internalization than etorphine (Zhang, J., Ferguson, S. S., Barak, L. S., Bodduluri, S. R., Laporte, S. A., Law, P. Y. & Caron, M. G., 1998, Proc Natl Acad Sci USA 95, 7157-62; Whistler, J. L. & von Zastrow, M., 1998, Proc Natl Acad Sci USA 95, 9914-9; and Bohn, L. M., Dykstra, L. A., Lefkowitz, R. J., Caron, M. G. & Barak, L. S., 2004, Mol Pharmacol 66, 106-12). Interestingly, each of these limitations can be overcome by overexpression of a GRK in cells suggesting that the agonist occupancy promotes different receptor conformations that result in differences in GRK-mediated regulation.

The β-arrestin proteins, namely β-arrestin-1 (βarr1) and β-arrestin-2 (βarr2) play an important role in GPCR desensitization. While the morphine-bound opioid receptor appears to be a poor substrate for βarr2 binding, a combination of both animal and cellular studies has revealed the importance of βarr2 in regulating this receptor. Mice that lack βarr2 display enhanced and prolonged morphine analgesia and display very little morphine tolerance (Bohn, L. M., Lefkowitz, R. J., Gainetdinov, R. R., Peppel, K., Caron, M. G. & Lin, F. T., 1999, Science 286, 2495-8; Bohn, L. M., Gainetdinov, R. R., Lin, F. T., Lefkowitz, R. J. & Caron, M. G., 2000, Nature 408, 720-3; Bohn, L. M., Lefkowitz, R. J. & Caron, M. G., 2002, J Neurosci 22, 10494-500; and Przewlocka, B., Sieja, A., Starowicz, K., Maj, M., Bilecki, W. & Przewlocki, R., 2002, Neurosci Lett 325, 107-10).

In contrast to their WT counterparts, these animals do not suffer from morphine-induced constipation or respiratory suppression (Raehal, K. M., Walker, J. K. & Bohn, L. M., 2005, J Pharmacol Exp Ther 314, 1195-201). Cellular studies suggest that the morphine-bound receptor has a preference for interacting with βarr2 over βarr1 and βarr1-KO mice do not display enhanced analgesic responses to morphine (Bohn, L. M., Dykstra, L. A., Lefkowitz, R. J., Caron, M. G. & Barak, L. S., 2004, Mol Pharmacol 66, 106-12). Taken together, these studies indicate that the morphine-bound μOR is poorly phosphorylated and has a low affinity for βarr2. Despite its low affinity, the βarr2 interaction plays a critical role in regulating the morphine-bound μOR and determining the extent of morphine analgesia and tolerance.

Currently, there is a need for new opioid receptor ligands that have fewer side effects than known ligands. Such ligands would be useful for the treatment of diseases and conditions associated with the activity of opioid receptors. Such ligands would also be useful as pharmacological tools for the further study of the physiological processes associated with opioid receptor structure and function.

### SUMMARY OF THE INVENTION

The invention provides novel opioid ligands. Accordingly, in one embodiment the invention provides a compound of the invention which is a compound of formula I:

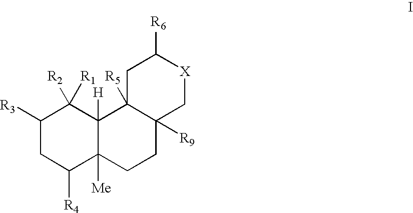

wherein:

R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;

R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;

R₄ is H, hydroxymethyl, (C₁-C₆)alkyl, (C₁-C₆)alkoxymethyl, carboxy, (C₁-C₆)alkoxycarbonyl or R_{d}RₑNC(═O)—;

R₅ is H or (C₁-C₆)alkyl;

R₆ is (C₁-C₆)alkyl, (C₁-C₆)cycloalkyl, aryl, Het, carboxy, RⱼRₖNC(═O)—, or heteroaryl;

R₉ is H or (C₁-C₆)alkyl;

X is —O—, —S—, or —NRₐ—;

each Rₐ is independently H, (C₁-C₆)alkyl, aryl, heteroaryl, aryl(C₁-C₆)alkyl, or heteroaryl(C₁-C₆)alkyl;

each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

each R_{c} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;

each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;

each R_{g} is independently (C₁-C₆)alkyl;

each Rₕ is independently H, (C₁-C₆)alkyl, fluoro, or chloro;

each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;

each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (R_{b})₃C(═NR_{d})—, or R_{b}S(═O)₂—;

each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and

each R_{q} is independently H, (C₁-C₆)alkyl, (C₂-C₆)alkenyl, aryl, heteroaryl, aryl(C₁-C₆)alkyl, or heteroaryl(C₁-C₆)alkyl;

wherein any aryl or heteroaryl of R₃, R₆, and Rₐ-Rₑ, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;

each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and

wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;

or a pharmaceutically acceptable salt thereof.

In another embodiment the invention provides a compound of the invention which is a compound of formula II:

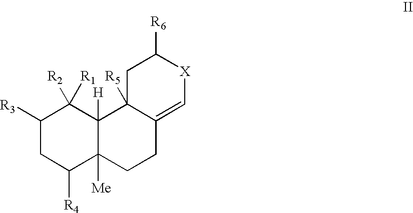

wherein:

R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;

R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;

R₄ is H, hydroxymethyl, (C₁-C₆)alkyl, (C₁-C₆)alkoxymethyl, carboxy, (C₁-C₆)alkoxycarbonyl or R_{d}RₑNC(═O)—;

R₅ is H or (C₁-C₆)alkyl;

R₆ is (C₁-C₆)alkyl, (C₁-C₆)cycloalkyl, aryl, Het, carboxy, RⱼRₖNC(═O)—, or heteroaryl;

X is —O—, —S—, or —NRₐ—;

each Rₐ is independently H, (C₁-C₆)alkyl, aryl, heteroaryl, aryl(C₁-C₆)alkyl, or heteroaryl(C₁-C₆)alkyl;

each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

each Rₑ is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;

each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;

each R_{g} is independently (C₁-C₆)alkyl;

each Rₕ is independently H, (C₁-C₆)alkyl, fluoro, or chloro;

each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;

each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (R_{b})₃C(═NR_{d})—, or R_{b}S(═O)₂—;

each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and

each R_{q} is independently H, (C₁-C₆)alkyl, (C₂-C₆)alkenyl, aryl, heteroaryl, aryl(C₁-C₆)alkyl, or heteroaryl(C₁-C₆)alkyl;

wherein any aryl or heteroaryl of R₃, R₆, and Rₐ-Rₑ, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;

each Rₜ is independently H, (C₁-C₆)alkenyl, (C₂-C₆)alkenyl, aryl, heteroaryl, aryl(C₁-C₆)alkyl, Het, Het(C₁-C₆)alkyl, or heteroaryl(C₁-C₆)alkyl;

wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ, and Rᵥ, are each independently H or (C₁-C₆)alkyl; and

wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;

or a pharmaceutically acceptable salt thereof.

In another embodiment the invention provides a compound of the invention which is a compound of formula III:

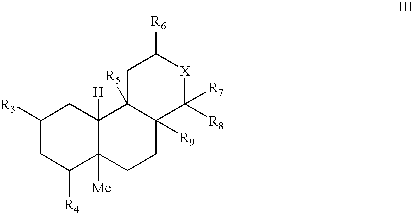

wherein:

R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;

R₄ is H, hydroxymethyl, (C₁-C₆)alkyl, (C₁-C₆)alkoxymethyl, carboxy, (C₁-C₆)alkoxycarbonyl or R_{d}RₑNC(═O)—;

R₅ is H or (C₁-C₆)alkyl;

R₆ is (C₁-C₆)alkyl, (C₁-C₆)cycloalkyl, aryl, Het, carboxy, RⱼRₖNC(═O)—, or heteroaryl;

R₇ and R₈ taken together are oxo (═O), thioxo (═S), or ═NRₐ;

R₉ is H or (C₁-C₆)alkyl;

X is —O—, —S—, or —NRₐ—;

each Rₐ is independently H, (C₁-C₆)alkyl, aryl, heteroaryl, aryl(C₁-C₆)alkyl, or heteroaryl(C₁-C₆)alkyl;

each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

each R_{d} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;

each R_{d} and R_{d} is independently H, (C₁-C₆)alkyl, (C₁-C₆)alkenyl, aryl, heteroaryl, aryl(C₁-C₆)alkyl, or heteroaryl(C₁-C₆)alkyl;

each R_{g} is independently (C₁-C₆)alkyl;

each Rₕ is independently H, (C₁-C₆)alkyl, fluoro, or chloro;

each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;

each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}R_{d}NC(═O)—, (R_{b})₃C(═NR_{d})—, or R_{b}S(═O)₂—;

each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and

each R_{q} is independently H, (C₁-C₆)alkyl, (C₂-C₆)alkenyl, aryl, heteroaryl, aryl(C₁-C₆)alkyl, or heteroaryl(C₁-C₆)alkyl;

wherein any aryl or heteroaryl of R₃, R₆, and Rₐ-R_{d}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;

each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and

wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;

or a pharmaceutically acceptable salt thereof.

In another embodiment the invention provides a compound of the invention which is a compound of formula IV:

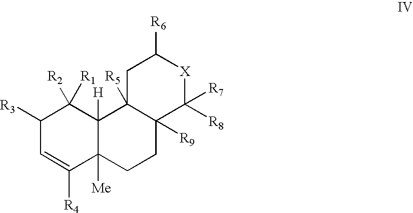

wherein:

R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;

R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;

R₄ is H, hydroxymethyl, (C₁-C₆)alkyl, (C₁-C₆)alkoxymethyl, carboxy, (C₁-C₆)alkoxycarbonyl or R_{d}RₑNC(═O)—;

R₅ is H or (C₁-C₆)alkyl;

R₆ is (C₁-C₆)alkyl, (C₁-C₆)cycloalkyl, aryl, Het, carboxy, RⱼRₖNC(═O)—, or heteroaryl;

R₇ and R₈ taken together are oxo (═O), thioxo (═S), or ═NRₐ;

R₉ is H or (C₁-C₆)alkyl;

X is —O—, —S—, or —NRₐ—;

each Rₐ is independently H, (C₁-C₆)alkyl, aryl, heteroaryl, aryl(C₁-C₆)alkyl, or heteroaryl(C₁-C₆)alkyl;

each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

each R_{c} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;

each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;

each R_{g} is independently (C₁-C₆)alkyl;

each Rₕ is independently H, (C₁-C₆)alkyl, fluoro, or chloro;

each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;

each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (Rₕ)₃C(═NR_{d})—, or R_{b}S(═O)₂—;

each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and

each R_{q} is independently H, (C₁-C₆)alkyl, (C₂-C₆)alkenyl, aryl, heteroaryl, aryl(C₁-C₆)alkyl, or heteroaryl(C₁-C₆)alkyl;

wherein any aryl or heteroaryl of R₃, R₆, and Rₐ, Rₑ, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;

each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and

wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;

or a pharmaceutically acceptable salt thereof.

In another embodiment the invention provides a pharmaceutical composition comprising a compound of the invention and a pharmaceutically acceptable diluent or carrier.

In another embodiment the invention provides a method for modulating the activity of an opioid receptor comprising contacting the receptor (in vivo or in vivo) with an effective modulatory amount of a compound of the invention.

In another embodiment the invention provides a therapeutic method for treating a disease or condition in an animal wherein the activity of an opioid receptor is implicated and modulation of the action of the receptor is desired (e.g. pain, drug addiction, or alcohol addiction) comprising administering to the animal, an effective amount of a compound of the invention.

In another embodiment the invention provides a compound of the invention for use in medical therapy.

In another embodiment the invention provides the use of a compound of the invention to prepare a medicament useful for the treatment of a disease or condition in a mammal wherein the activity of an opioid receptor is implicated and modulation of the action of the receptor is desired.

In another embodiment the invention provides a compound of formula I, II, III, or IV as described herein that comprises or that is attached directly or through a linker to one or more detectable groups; or a pharmaceutically acceptable salt thereof.

In another embodiment the invention provides a therapeutic method for treating a disease or condition in an animal wherein the activity of a mu-opioid receptor is implicated and modulation of the action of the receptor without recruiting beta-arrestins is desired comprising administering to the animal, an effective amount of a compound of formula V:

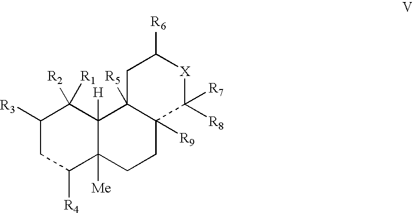

wherein:

R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;

R₃ is aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, RₓO—, RₓC(═O)O—, RₓC(═S)O—, R_{y}R_{z}NC(═O)O—, R_{y}R_{z}N, RₓC(═O)N(H)—, R_{w}S(═O)₂N(H)—, or R_{w}S(═O)₂O—; each R_{w} is independently aryl, heteroaryl, aryl(C₁-C₆)alkyl, or heteroaryl(C₁-C₆)alkyl; each Rₓ is independently aryl, heteroaryl, aryl(C₁-C₆)alkyl, or heteroaryl(C₁-C₆)alkyl; R_{y} is aryl, heteroaryl, aryl(C₁-C₆)alkyl, or heteroaryl(C₁-C₆)alkyl; and R_{z} is independently H, (C₁-C₆)alkyl, (C₁-C₆)alkenyl, aryl, heteroaryl, aryl(C₁-C₆)alkyl, or heteroaryl(C₁-C₆)alkyl;

R₄ is H, hydroxymethyl, (C₁-C₆)alkyl, (C₁-C₆)alkoxymethyl, carboxy, (C₁-C₆)alkoxycarbonyl or R_{y}R_{z}NC(═O)—;

R₅ is H or (C₁-C₆)alkyl;

R₆ is (C₁-C₆)alkyl, (C₁-C₆)cycloalkyl, aryl, Het, carboxy, RⱼRₖNC(═O)—, or heteroaryl;

each bond represented by - - - is a single bond or a double bond;

R₇ and R₈ taken together are oxo (═O), thioxo (═S), or ═NRₐ, or R₈ is absent when the bond represented by - - - is a double bond;

R₉ is H or (C₁-C₆)alkyl, or R₉ is absent when the bond represented by - - - is a double bond;

X is —O—, —S—, or —NRₐ—;

each Rₐ is independently H, (C₁-C₆)alkyl, aryl, heteroaryl, aryl(C₁-C₆)alkyl, or heteroaryl(C₁-C₆)alkyl;

each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;

each R_{q} is independently H, (C₁-C₆)alkyl, (C₂-C₆)alkenyl, aryl, heteroaryl, aryl(C₁-C₆)alkyl, or heteroaryl(C₁-C₆)alkyl;

wherein any aryl or heteroaryl of R₃, R₆, and Rₐ, Rⱼ-Rₖ, R_{q}, and R_{w}-R_{z}, is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;

each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;

wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and

wherein any Het of R₃, R₆, R_{w}, Rₓ, Rⱼ-Rₖ, and R_{q}, is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;

or a pharmaceutically acceptable salt thereof.

In another embodiment the invention provides a therapeutic method for treating a disease or condition in an animal wherein the activity of a mu-opioid receptor is implicated and modulation of the action of the receptor without promoting internalization of the receptor is desired comprising administering to the animal, an effective amount of a compound of formula V or a pharmaceutically acceptable salt thereof as described herein.

In another embodiment the invention provides a therapeutic method for treating a disease or condition in an animal wherein the activity of a mu-opioid receptor is implicated and modulation of the action of the receptor without promoting desensitization of the receptor is desired comprising administering to the animal, an effective amount of a compound of formula V or a pharmaceutically acceptable salt thereof as described herein.

In another embodiment the invention provides a therapeutic method for treating a disease or condition in an animal wherein the activity of a mu-opioid receptor is implicated and modulation of the action of the receptor without promoting robust phosphorylation of the receptor is desired comprising administering to the animal, an effective amount of a compound of formula V or a pharmaceutically acceptable salt thereof as described herein.

In another embodiment the invention provides a therapeutic method for treating a disease or condition in an animal wherein the activity of a mu-opioid receptor is implicated and modulation of the action of the receptor without promoting βarr2-GFP translocation is desired comprising administering to the animal, an effective amount of a compound of formula V or a pharmaceutically acceptable salt thereof as described herein.

In another embodiment the invention provides a therapeutic method for producing an analgesic effect in an animal without promoting tolerance comprising administering to the animal, an effective amount of a compound of formula V or a pharmaceutically acceptable salt thereof as described herein.

In another embodiment the invention provides a therapeutic method for treating a disease or condition in an animal wherein the activity of a MAP kinase is implicated and modulation of the action of the MAP kinase is desired comprising administering to the animal, an effective amount of a compound of formula V or a pharmaceutically acceptable salt thereof as described herein.

In another embodiment the invention provides a therapeutic method for treating a disease or condition in an animal wherein the activity of an ERIC kinase is implicated and modulation of the action of the ERIC kinase is desired comprising administering to the animal, an effective amount of a compound of formula V or a pharmaceutically acceptable salt thereof as described herein.

The invention also provides a method for binding a compound of the invention to mammalian tissue comprising opioid receptors, in vivo or in vitro, comprising contacting the tissue with an amount of a compound of the invention effective to bind to said receptors. Tissue comprising a compound of the invention bound to opioid receptor sites can be used to measure the selectivity of test compounds for specific receptor subtypes, or can be used as a tool to identify potential therapeutic agents for the treatment of diseases or conditions associated with opioid receptor activity, by contacting said agents with said ligand-receptor complexes, and measuring the extent of displacement of the ligand and/or binding of the agent.

The invention also provides processes and intermediates disclosed herein that are useful for preparing compounds of the invention or salts thereof. Certain compounds of the invention are useful as intermediates for preparing other compounds of the invention.

### BRIEF DESCRIPTION OF THE FIGURES

FIG. 1. Shows the structures of morphine (2), fentanyl (3), cyclazocine (4), SNC 80 (5), U50,488H (6), and 3FLB (7).

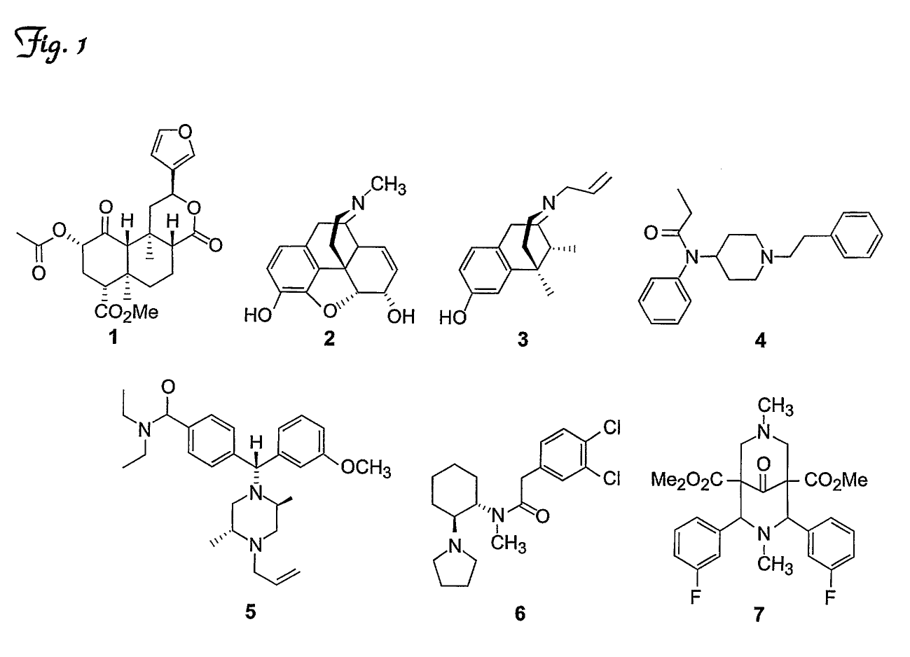

FIG. 2. Compound Vb (also referred to herein as herkinorin) promotes ERK1/2 phosphorylation. Compound Vb induces ERK1&2 phosphorylation in HEK-293 cells stably expressing an hemagglutinin (HA-N-terminus) tagged mouse μOR. Compound Vb stimulation was for 10 minutes at the concentrations indicated. Pretreatment with naloxone (10 μM) during the 30 min serum starvation blocked Compound Vb-induced ERK activation. Phopsho-ERK1/2 bands were analyzed by densitometry and normalized to total ERK levels (lower bands) and densitometry is presented as fold stimulation (mean±SEM) over saline treated controls. ** p<0.01 vs. Saline; *** p<0.001 vs. Saline, Student's t test (n=3-5).

FIG. 3. Mu opioid receptor phosphorylation at serine 375 following agonist treatment. HEK-293 cells stably expressing HA-tagged mu opioid receptor were treated with saline, 1 μM DAMGO, 10 μM Morphine, or 10 μM Compound Vb (herkinorin) for 10 minutes. The receptor was immunoprecipitated from cell lysates using an anti-HA antibody-agarose bead complex. Representative western blots using antibodies that recognize the μOR phosphorylated at serine 375 (top) or the total μOR (C-terminal antibody) from the same blot (bottom) are shown. Densitometric analysis of 3 independent experiments performed in duplicate or triplicate were normalized to total receptor per lane and expressed as % stimulation over saline control for each blot. Data are presented as the mean±S.E.M. **p<0.0001; 4-p<0.005 vs. Saline; ^{##}p<0.0001 vs. DAMGO Student's t test (n=5-8).

FIG. 4. Agonist-induced βarr2-GFP translocation to μOR in HEK-293 cells. HEK-293 cells transiently transfected with HA-μOR were imaged in real time following agonist treatment at room temperature. The cytosolic distribution of βarr2-GFP is shown in the untreated cells in the top left panels. A. βarr2-GFP translocation to μOR in HEK-293 cells. DAMGO (1 μM) treatment leads to βarr2-GFP translocation within 5 min (white arrow-punctuate accumulation at membrane) while morphine (10 μM, 10 min) does not. Compound Vb (2 μM, 10 min) does not induce βarr2-GFP translocation. B. βarr2-GFP translocation to μOR in HEK-293 cells overexpressing GRK2. Compound Vb does not promote βarr2-GFP translocation at 2 μM after 10 min nor at 100 μM after 30 min. The same cells were treated with morphine (10 μM, 10 min) and βarr2-GFP translocates demonstrating that these cells do overexpress GRK2 as morphine would not be able to induce visible translocation otherwise.

FIG. 5. Agonist-induced internalization of μOR-YFP in HEK-293 cells. A. HEK-293 cells were transiently transfected with mouse μOR (2 μg cDNA) tagged at the C-terminus with yellow fluorescent protein (YFP). Cells were treated with the agonists indicated. Internalization can be seen after DAMGO treatment as indicated by the appearance of donut-like intracellular vesicles (white arrow) and the disappearance of membrane receptor localization as seen in the basal panel. Compound Vb and morphine do not induce receptor internalization. B. DAMGO, but not Compound Vb, leads to a loss of cell surface expression following 2 hours of drug treatment. Two-way ANOVA analysis reveals that the curves differ (P<0.001) and that the DAMGO treated cells display less surface receptors at each time point as determined by Bonferoni post-hoc analysis (p<0.001 at each time point). C. Agonist-induced μOR-YFP internalization in cells overexpressing GRK2. Experiments were performed as described in FIG. 4A with the addition of co-transfecting 5 μg GRK2 cDNA. Morphine induces a redistribution of membrane associated μOR-YFP to intracellular vesicle (white arrows). Compound Vb does not. D. Agonist induced μOR1-D-GFP internalization. μOR1-D-GFP (2 μg cDNA) was transiently transfected into HEK-293 cells. Morphine treatment internalizes the receptor as indicated by the appearance of intracellular vesicles (white arrows) while Compound Vb does not. Experiments were performed on at least 3 separate transfections; representative cells are shown.

FIG. 6. Compound Vb-induced antinociception in the rat formalin paw withdrawal assay. A. Acute effects of Compound Vb (˜0.04 mg/kg s.c. paw) or vehicle injected 5 minutes prior to injection of 1.25% formalin into the plantar surface of the hind paw. Naloxone (5 mg/kg s.c.) was administered 30 minutes prior to formalin treatment; nor-binaltorphimine (nor-BNI) (5 mg/kg s.c.) was administered 24 hours prior to the formalin treatment. Vehicle vs. Compound Vb: F_{(1,120)}=49.35, p<0.0001; Compound Vb vs. Compound Vb+Naloxone: F_{(1,120)}=4.24, p=0.0416; Compound Vb vs. Compound Vb+Nor-BNI: F_{(1,120)}=65.01, p<0.0001, two-way ANOVA, n=6 rats/group. B. Chronic Compound Vb treatment (5 days) did not result in the development of antinociceptive tolerance. Vehicle Day 1 vs. Compound Vb Day 1: F_{(1,60)}=19.77, p<0.0001; Vehicle Day 1 vs. Vehicle Day 5: F_{(1,84)}=0.12, p=0.7265; Compound Vb Day 1 vs. Compound Vb Day 5: F_{(1,96)}=7.81, (p=0.0063); Vehicle Day 5 vs. Compound Vb Day 5: F_{(1,120)}=167.31, p<0.0001, two-way ANOVA, n=3-6 rats/group. C. Total number of flinches in the second phase of the formalin test comparing Day 1 versus Day 5 following Compound Vb daily administration. Compound Vb treatment leads to less flinches than vehicle on Day 1 (***P<0.001) and on Day 5 (***p<0.001). Vehicle on Day 1 does not differ from Vehicle on Day 5; Compound Vb on Day 1 does not differ from Compound Vb on Day 5 (p>0.05), one-way ANOVA analysis of variance; Bonferroni's multiple comparison test.

### DETAILED DESCRIPTION

The following definitions are used, unless otherwise described: halo is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(Y) wherein Y is absent or is H, O, (C₁-C₄)alkyl, phenyl or benzyl, as well as a radical of an ortho-fused bicyclic heterocycle of about eight to ten ring atoms derived there from, particularly a benz-derivative or one derived by fusing a propylene, trimethylene, or tetramethylene diradical thereto. “Het” includes a mono or bicyclic saturated or partially unsaturated ring system comprising about 4 to about 12 atoms selected from carbon, O, S, and N. Examples of “Het” include dihydrofuran, tetrahydrofuran, pyrazoline, piperidine, morpholine, thiomorpholine, piperazine, indoline, isoindoline, pyrazolidine, imidazoline, imidazolidine, pyrroline, pyrrolidine, chroman, and isochroman.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine opioid receptor binding and modulatory activity using the standard tests described herein, or using other similar tests which are well known in the art.

As used herein the term “without recruiting beta-arrestins,” means that the compounds typically cause less recruitment of beta-arrestins than is seen with other analgesics, such as those that operate at the mu-receptor (e.g. enkephalin analogs). Typically, the compounds of the invention do not significantly produce recruitment of beta-arrestins. Accordingly, they do not cause significant internalization of the mu-receptor.

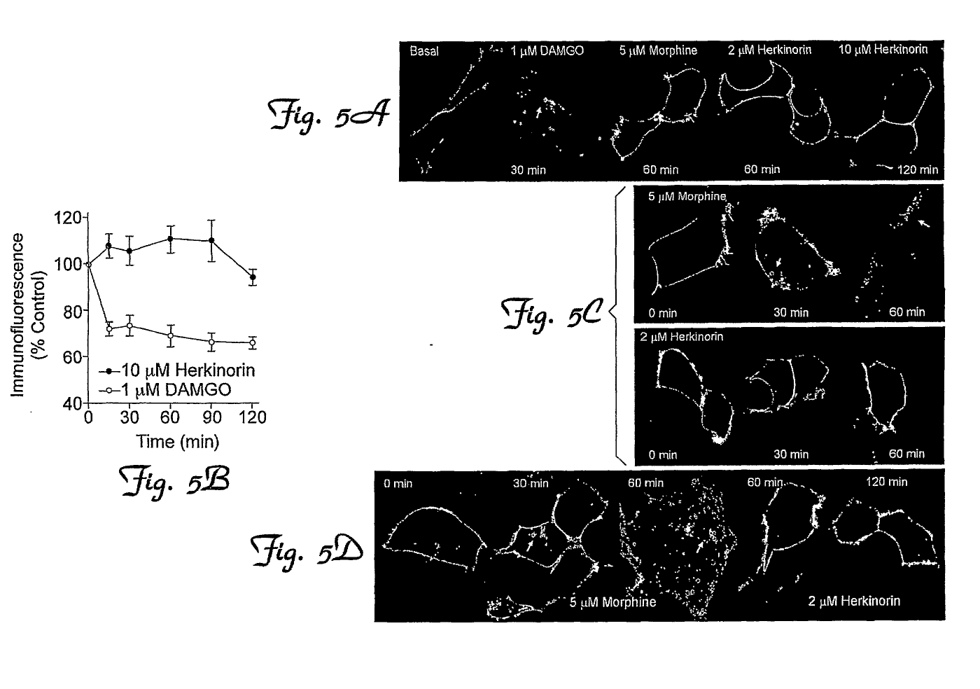

As used herein the term “without promoting internalization of the receptor” means that the compounds typically cause less internalization of the mu-receptor than is seen with other analgesics (e.g. enkephalin analogs). Typically the compounds cause less than about 30% internalization of the mu-receptor, as illustrated in FIG. 5. In one specific embodiment of the invention the compounds cause less than about 20% internalization of the mu-receptor. In another specific embodiment of the invention, the compounds cause less than about 10% internalization of the mu-receptor. In another specific embodiment of the invention, the compounds cause less than about 5% internalization of the mu-receptor.

As used herein the term “without promoting desensitization” means that the compounds typically cause less desensitization of the mu-receptor than is seen with other analgesics. Desensitization can be caused by different mechanisms, for example by receptor internalization.

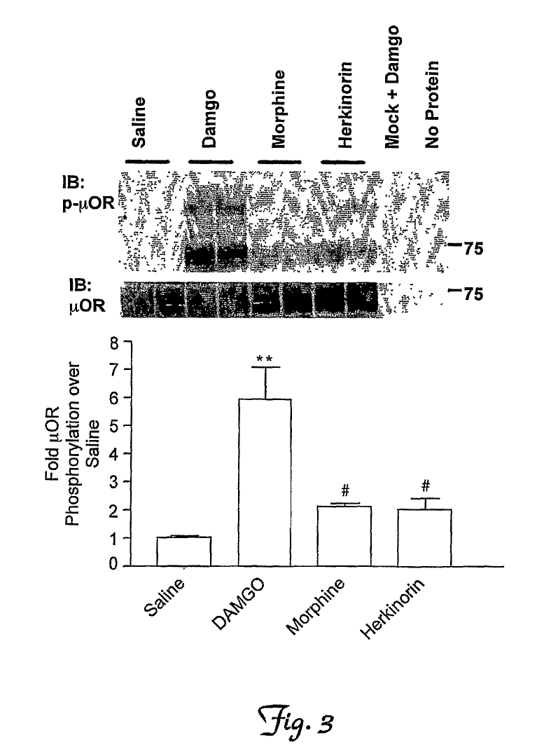

As used herein the term “without promoting robust phosphorylation of the receptor,” means the compound causes less than about 5 fold phosphorylation of the receptor over a saline control (see FIG. 3). In one specific embodiment of the invention, the compounds cause less than about 4 fold phosphorylation of the receptor over a saline control. In another specific embodiment of the invention, the compounds cause less than about 3 fold phosphorylation of the receptor over a saline control. In another specific embodiment of the invention, the compounds cause less than about 2 fold phosphorylation of the receptor over a saline control.

As used herein the term “without promoting βarr2-GFP translocation” means that the compounds typically cause less βarr2-GFP translocation than other analgesics (e.g. enkephalin analogs).

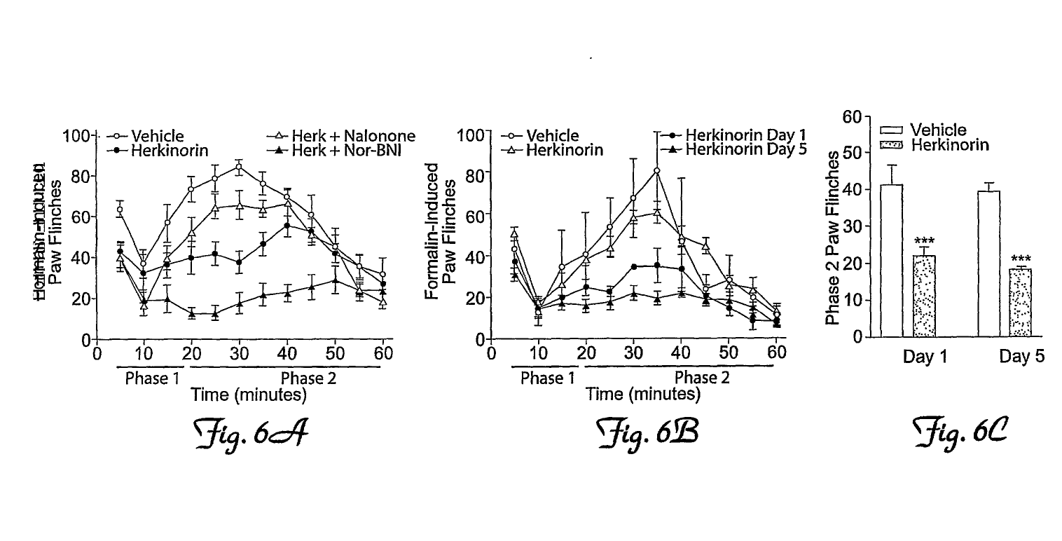

As used herein the term “without promoting tolerance,” Means that the compounds typically produce less tolerance than other analgesics (e.g. morphine). For example, in one embodiment of the invention, at least about 50% of the analgesic activity of the compound is maintained following once a day dosing for 10 days (see FIG. 6). In another embodiment of the invention, at least about 60% of the analgesic activity of the compound is maintained following once a day dosing for 10 days. In another embodiment of the invention, at least about 75% of the analgesic activity of the compound is maintained following once a day dosing for 10 days. In another embodiment of the invention, at least about 90% of the analgesic activity of the compound is maintained following once a day dosing for 10 days.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Specific Values

A specific value for R₁ is hydroxy, (C₁-C₆)alkoxy, or (C₁-C₆)alkanoyloxy and R₂ is H.

A specific value for R₁ and R₂ taken together is oxo (═O), thioxo (═S), or ═NRₐ.

Another specific value for R₁ and R₂ taken together is oxo (═O).

A specific value for R₃ is H, hydroxy, (C₁-C₆)alkyl, (C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, formyloxy, acetoxy, R_{c}C(═O)O—, (R_{g})₃SiO—, R_{d}RₑNC(═O)O—, (Rₕ)₃C(═NR_{d})O—, or R_{b}S(═O)₂O—.

Another specific value for R₃ is hydroxy, (C₁-C₆)alkoxy, aryloxy, heteroaryloxy, aryl(C₁-C₆)alkoxy, heteroaryl(C₁-C₆)alkoxy, formyloxy, R_{c}C(═O)O—, or R_{b}S(═O)₂O—.

Another specific value for R₃ is formyloxy, R_{c}C(═O)O—, or R_{b}S(═O)₂O—.

Another specific value for R₃ is propanoyloxy, isobutanoyloxy, methacryloyloxy, methoxyoxalyloxy, benzoyloxy, trimethylsilyloxy, imidazole-1-ylthiocarbonyloxy, methoxymethoxy, aminocarbonyloxy, butanoyloxy, pentanoyloxy, 1-bromobenzoyloxy, 2-bromobenzoyloxy, 3-bromobenzoyloxy, 4-methoxybenzoyloxy, 4-nitrobenzoyloxy, phenylsulfonyloxy, 4-methylphenylsulfonyloxy, 4-methoxyphenylsulfonyloxy, 4-bromophenylsulfonyloxy, (3-pyridylcarbonyloxy, methylsulfonyloxy, hydroxy, 1-imino-2,2,2-trichloroethoxy, phenylaminocarbonyloxy, alkylaminocarbonyloxy, 3,4-dichlorobenzoyloxy, bromo, azido, amino, acetylamino, phenylcarbonylamino, methylsulfonylamino, phenylsulfonylamino, or benzoyloxy.

Another specific value for R₃ propanoyloxy, methylsulfonyloxy, or benzoyloxy.

A specific value for R₄ is hydroxymethyl, (C₁-C₆)alkoxymethyl, carboxy, (C₁-C₆)alkoxycarbonyl; or R_{d}NC(═O)—.

Another specific value for R₄ is carboxy, (C₁-C₆)alkoxycarbonyl; or R_{d}RₑNC(═O)—.

Another specific value for R₄ is methoxycarbonyl.

A specific value for R₅ is H.

Another specific value for R₅ is methyl.

A specific value for R₆ is aryl or heteroaryl, optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RₑR_{f}N.

Another specific value for R₆ is phenyl, thienyl, furanyl, pyrrolyl, or pyridyl, optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RₑR_{f}N.

Another specific value for R₆ is 3-furyl.

A specific value for R₇ and R₈ taken together is oxo.

A specific value for X is —O—.

A specific value for Rₐ is H, methyl, ethyl, phenyl, thienyl, furanyl, pyrrolyl, pyridyl, benzyl, phenethyl, thienylmethyl, furanylmethyl, pyrrolylmethyl, or pyridylmethyl.

A specific value for R_{b} is independently H, (C₁-C₆)alkyl, (C₂-C₆)alkenyl, aryl, heteroaryl, aryl(C₁-C₆)alkyl, or heteroaryl(C₁-C₆)alkyl.

A specific value for R_{c} is independently H, ethyl, phenyl, thienyl, furanyl, pyrrolyl, pyridyl, benzyl, phenethyl, thienylmethyl, furanylmethyl, pyrrolylmethyl, or pyridylmethyl.

A specific value for R_{d} is H, methyl, ethyl, phenyl, thienyl, furanyl, pyrrolyl, pyridyl, benzyl, phenethyl, thienylmethyl, furanylmethyl, pyrrolylmethyl, or pyridylmethyl.

A specific value for Rₑ is H, methyl, ethyl, phenyl, thienyl, furanyl, pyrrolyl, pyridyl, benzyl, phenethyl, thienylmethyl, furanylmethyl, pyrrolylmethyl, or pyridylmethyl.

A specific compound is a compound of formula (Ia):

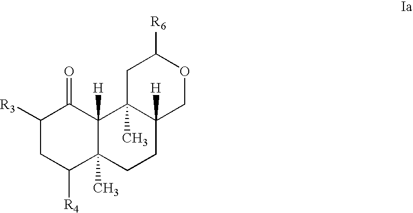

wherein R₃, R₄, and R₆ have any of the values defined herein; or a pharmaceutically acceptable salt thereof.

A specific compound is a compound of formula (IIa):

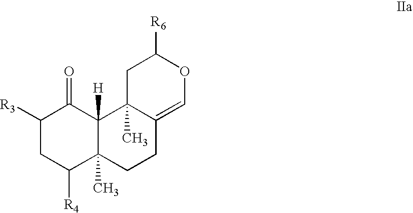

wherein R₃, R₄, and R₆ have any of the values defined herein; or a pharmaceutically acceptable salt thereof.

A specific compound is a compound of formula (IIIa):

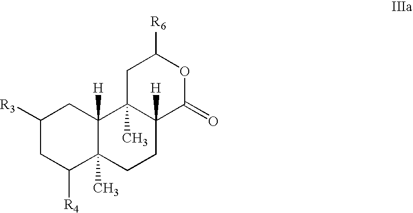

wherein R₃, R₄, and R₆ have any of the values defined herein; or a pharmaceutically acceptable salt thereof.

A specific compound is a compound of formula (IVa):

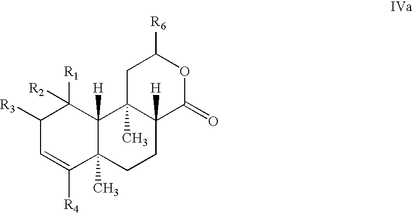

wherein R₁ is H, hydroxy, (C₁-C₆)alkoxy, or (C₁-C₆)alkanoyloxy and R₂ is H; and R₃, R₄, and R₆ have any of the values defined herein; or a pharmaceutically acceptable salt thereof.

A specific compound is a compound of formula (Va):

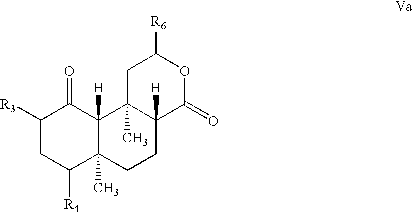

wherein R₃, R₄, and R₆ have any of the values defined herein, or a pharmaceutically acceptable salt thereof.

A specific compound is a compound of formula (Vb):

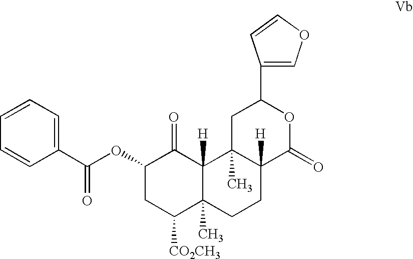

or a pharmaceutically acceptable salt thereof.

In one embodiment of the invention the compound of Formula (I) is not a compound of the following formula:

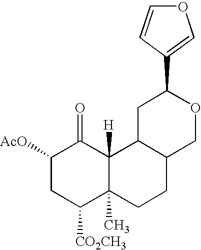

In one embodiment of the invention the compound of Formula (II) is not a compound of the following formula:

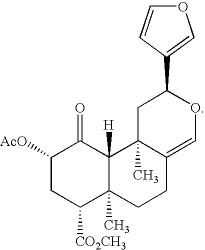

In one embodiment of the invention the compound of Formula (II) is not a compound of the following formula:

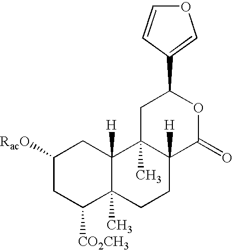

wherein R_{ac} is H or CH₃C(═O)—.

In one embodiment of the invention the compound of Formula (IV) is not a compound of the following formula:

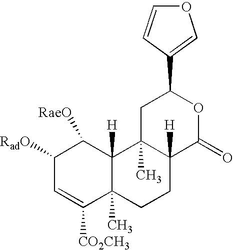

wherein R_{ad} is H or CH₃C(═O)—; and Rₐₑ is H or CH₃C(═O)—.

### Labeled Compounds

Specific compounds of the invention also include compounds of formulae I-Vb that comprise or that are linked to one or more detectable groups or isotopes. Such detectable compounds may be used as imaging agents or as probes for evaluating opioid receptor structure and function. For example, one or more detectable groups can be incorporated into the core of the compound, or can be attached to the compound directly, through a linking group, or through a chelating group. Suitable detectable groups include deuterium, tritium, iodine-125, iodine-131, iodine-123, astatine-210, carbon-11, carbon-14, nitrogen-13, or fluorine-18. Additionally, groups such as Tc-99m and Re-186 can be attached to a linking group or bound by a chelating group which is then attached to the compound directly or by means of a linker. Suitable radiolabeling techniques are routinely used in radiopharmaceutical chemistry.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound is conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

### General Experimental

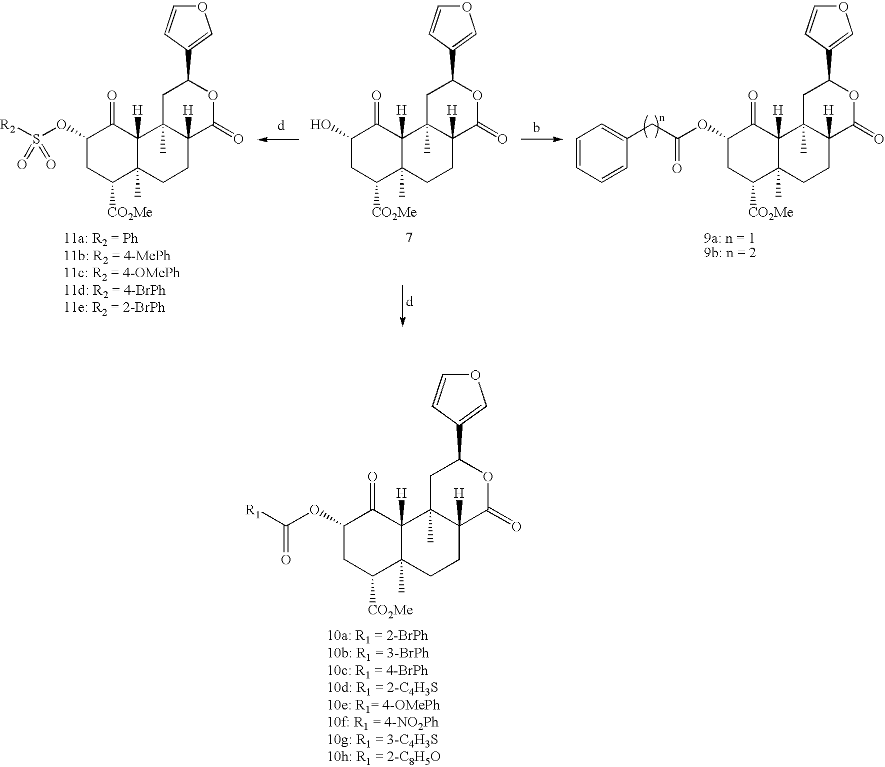

The binding affinities of representative compounds of the invention at opioid receptors can be determined using [¹²⁵I]IOXY as radioligand (see Ni, Q.; Xu, H.; Partilla, J. S.; de Costa, B. R.; Rice, K. C. et al., Peptides 1993, 14, 1279-1293; and de Costa, B. R.; Iadarola, M. J.; Rothman, R. B.; Berman, K. F.; George, C. et al., J. Med. Chem. 1992, 35, 2826-2835).

The functional activity of the compounds can further be evaluated using a [³⁵S]GTPγS assay (Xu, H.; Hashimoto, A.; Rice, K. C.; Jacobson, A. E.; Thomas, J. B. et al., Synapse 2001, 39, 64-69).

The invention will now be illustrated by the following non-limiting Examples.

### EXAMPLES

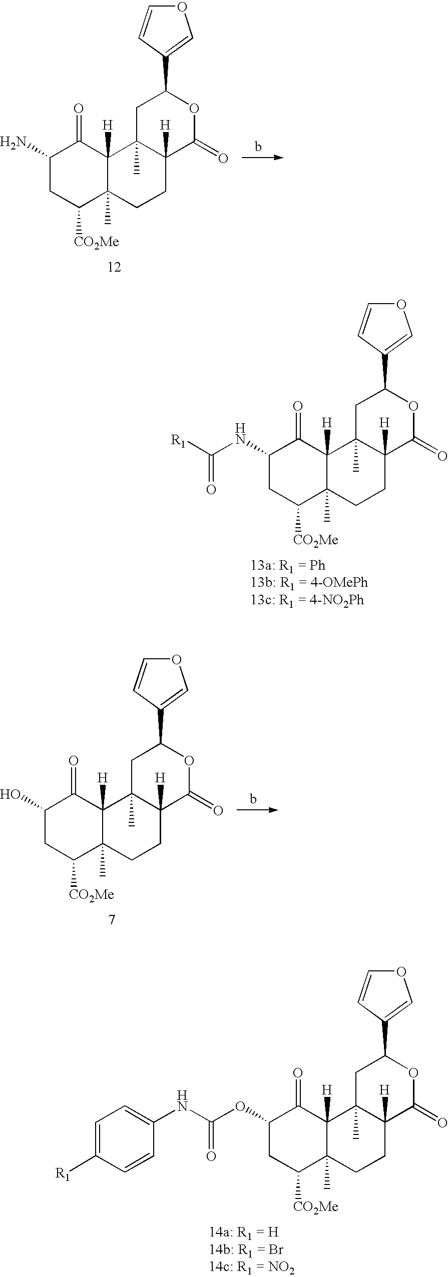

### Example 1

### Isolation of Salvinorin A (1)

Dried Salvia divinorum leaves (1.5 kg), obtained commercially from Ethnogens.com, were ground to a fine powder and percolated with acetone (5×4 L). The acetone extract was concentrated under reduced pressure to afford a crude green gum (93 g), which was subjected to column chromatography on silica gel with elution in n-hexanes containing increasing amounts EtOAc. Fractions eluting in 20% n-hexanes/EtOAc contained salvinorin A (TLC) and other minor diterpenes and some pigmented material. These fractions were pooled and concentrated in vacuo to give a green gum (24 g). A mixture of the crude green gum, acetic anhydride (50 mL, 530 mmol) and DMAP (0.2 g) in CH₂Cl₂ (250 mL) was stirred at RT overnight. The CH₂Cl₂ solution was washed sequentially with 1N HCl (2×500 mL), 2N NaOH (100 mL), and H₂O (2×100 mL). The CH₂Cl₂ solution was dried Na₂SO₄) and the solvent was removed under reduced pressure to afford a yellow-green gum (23 g). The resulting gum was subjected to column chromatography on silica gel. Elution was performed in 1000 mL aliquots of a mixture of n-hexanes/EtOAc in increments of 10% EtOAc with the final elution in neat EtOAc. Fractions eluting in 30% n-hexanes/EtOAc and subsequent fractions were pooled and the solvent was removed under reduced pressure affording salvinorin A (1 7.5 g, 0.5%) as a green powder, mp 235-238° C. (Lit.^{1,2} 240-242° C.).

### Example 2

(2S,4aS,6aR,7R,9S,10aS,10bR)-9-(Phenylacetyloxy)-2-(3-furanyl)-dodecahydro-6a,10b-dimethyl-4,10-dioxo-2H-naphtho[2,1-c]pyran-7-carboxylic acid methyl ester (9a). 9a was synthesized from 7 using phenylacetyl chloride as described for 8a to afford 70 mg (54%) of 9a as a white solid, mp 111-114° C.; ¹H NMR (CDCl₃): δ 1.10 (3H, s); 1.40 (3H, s); 1.41-1.52 (2H, m); 1.61 (1H, m); 1.73 (1H, m); 1.87 (1H, dd, J=3.0, 11.4); 2.10 (1H, dd, J=3.0, 13.2); 2.22 (1H, s); 2.31 (2H, m); 2.72 (1H, m); 3.72 (3H, s); 3.75 (2H, s); 5.17 (1H, dd, J=9.3, 9.3); 5.37 (1H, dd, J=4.8, 11.7); 6.36 (1H, dd, J=0.9, 1.5); 7.27-7.36 (6H, m); 7.38-7.42 (2H, m); ¹³C NMR (CDCl₃): δ 15.3, 16.6, 18.2, 30.9, 35.5, 38.2, 40.8, 42.1, 42.9, 51.3, 52.1, 53.6, 63.8, 72.0, 75.6, 108.8, 125.2, 127.4, 128.8, 129.6, 133.7, 139.9, 143.8, 171.0, 171.3, 171.8, 202.0. anal C, 68.74%; H, 6.36%; O, 24.93%; calcd for C₂₉H₃₂O₈, C, 68.49%; H, 6.34%; O, 25.17%.

### Example 3

(2S,4aS,6aR,7R,9S,10aS,10bR)-9-(3-Phenylpropionyl)-2-(3-furanyl)-dodecahydro-6a,10b-dimethyl-4,10-dioxo-2H-naphtho[2,1-c]pyran-7-carboxylic acid methyl ester (9b). 9b was synthesized from 7 as described for 8a using hydrocinnamoyl chloride to afford 69 mg (63%) of 9b as a white solid, mp 155-158° C.; ¹H NMR (CDCl₃): δ 1.14 (3H, s); 1.47 (3H, s); 1.52-1.74 (3H, m); 1.81 (1H, m); 2.09 (1H, dd, J=3.0, 11.1); 2.17 (1H, m); 2.19 (1H, s); 2.25-2.34 (2H, m); 2.53 (1H, dd, J=5.3, 13.7); 2.71-2.76 (1H, m); 2.79 (2H, t, J=7.5), 3.01 (2H, t, J=7.5); 3.74 (3H, s); 5.17 (1H, dd, J=10.1, 10.1); 5.55 (1H, dd, J=4.8, 11.7); 6.40, (1H, dd, J=0.9, 1.8), 7.20-7.26 (3H, m); 7.32 (2H, m); 7.43 (2H, m); ¹³C NMR (CDCl₃): 15.4, 16.6, 18.4, 31.0, 35.6, 35.7, 38.4, 42.3, 43.6, 51.6, 52.2, 53.8, 64.3, 72.2, 75.2, 77.4, 108.6, 125.4, 126.6, 128.5, 128.7, 139.7, 140.4, 143.9, 171.3, 171.7, 172.1, 202.1. anal. C, 69.23%; H, 6.59%; O, 24.23%; calcd for C₃₀H₃₄O₈, C, 68.95%; H, 6.56%; O, 24.49%.

### Example 4

(2S,4aS,6aR,7R,9S,10aS,10bR)-9-(2-Bromobenzoyloxy)-2-(3-furanyl)-dodecahydro-6a,10b-dimethyl-4,10-dioxo-2H-naphtho[2,1-c]pyran-7-carboxylic acid methyl ester (10a). 10a was synthesized as described for 8a from 7 using 2-bromobenzoyl chloride to afforded 70 mg (60%) of 10a as a white solid, mp 189-191° C.; ¹H NMR (CDCl₃): δ 1.15 (3H, s); 1.45 (3H, s); 1.59-1.67 (3H, m); 1.82 (1H, m); 2.11-2.16 (2H, m); 2.35 (1H, s); 2.40-2.51 (3H, m); 2.86 (1H, dd, J=8.4, 8.4); 3.73 (3H, s); 5.40-5.50 (2H, m); 6.39 (1H, m); 7.34-7.42 (4H, m); 7.69 (1H, dd, J=1.8, 7.4); 7.97 (1H, dd, J=2.4, 7.4). ¹³C NMR (CDCl₃): δ 15.4, 16.7, 18.4, 31.0, 25.7, 38.4, 42.4, 43.6, 51.6, 52.2, 53.8, 64.3, 72.3, 76.1, 108.6, 122.2, 125.4, 127.5, 131.1, 132.1, 133.3, 134.7, 139.7, 143.9, 165.1, 171.3, 171.7, 201.8. anal. C, 58.46%; H, 5.16%; O, 22.07%; calcd for C₂₈H₂₉BrO₈, C, 58.65%; H, 5.10%; O, 22.32%.

### Example 5

(2S,4aS,6aR,7R,9S,10aS,10bR)-9-(3-Bromobenzoyloxy)-2-(3-furanyl)-dodecahydro-6a,10b-dimethyl-4,10-dioxo-2H-naphtho[2,1-c]pyran-7-carboxylic acid methyl ester (10b). 10b was synthesized as described for 8a from 7 using 3-bromobenzoyl chloride to afford 75 mg (70%) of 10b as a white solid, mp 195-199° C.; ¹H NMR (CDCl₃): δ 1.17 (3H, s); 1.45 (3H, s); 1.28-1.69 (3H, m); 1.82 (1H, m); 2.10 (1H, dd, J=2.4, 11.1); 2.18 (1H, m); 2.29 (1H, s); 2.42-2.54 (3H, m); 2.83 (1H, dd, J=6.3, 10.5); 3.75 (3H, s); 5.39 (1H, dd, J=9.0, 9.0); 5.52 (1H, dd, J=5.1, 11.7); 6.38 (1H, m); 7.32-7.42 (3H, m); 7.72 (1H, ddd, J=1.2, 1.8, 7.8); 8.01 (1H, ddd, J=1.2, 1.2, 7.8); 8.21 (1H, dd, J=51.8, 1.8); ¹³C NMR (CDCl₃): δ 15.4, 16.7, 18.4, 31.1, 35.7, 38.4, 42.5, 43.7, 51.6, 52.3, 53.8, 64.4, 72.3, 76.0, 108.6, 122.8, 125.4, 128.7, 130.3, 131.2, 133.1, 136.7, 139.7, 143.9, 164.4, 171.3, 171.7, 201.7. anal. C, 58.54%; H, 5.18%; O, 22.15%; calcd for C₂₈H₂₉BrO₈, C, 58.65%; H, 5.10%; O, 22.32%.

### Example 6

Thiophene-2-carboxylic acid (2S,4aR,6aR,7R,9S,10aS,10bR)-7-carbomethoxy-2-(3-furanyl)dodecahydro-6a,10b-dimethyl-4,10-dioxo-2H-naphtho[2,1-c]pyran-9-yl ester (10d). A solution of 7 (80 mg, 0.21 mmol), 2-thiophene carbonyl chloride (0.11 mL, 2.73 mmol), Et₃N (0.38 mL, 2.73 mmol), pyridine (0.22 mL, 2.73 mmol) and a catalytic amount of DMAP in CH₂Cl₂ (40 mL) was stirred at room temperature overnight. The mixture was diluted with CH₂Cl₂ (30 mL) and washed with 2N HCl (2×30 mL), saturated NaHCO₃ (2×30 mL) and water (40 mL). The organic extract was then dried (Na₂SO₄), filtered and concentrated to give an oil which was purified by flash column chromatography (30-40% ethyl acetate/hexanes) to give 63 mg (60%) of 10d as a white solid, mp 143-145° C.; ¹H NMR (CDCl₃): δ 1.18 (3H, s); 1.48 (3H, s); 1.54-1.77 (3H, m); 1.84 (1H, dd, J=3.0, 10.2); 2.12 (1H, dd, J=2.2, 11.1); 2.19 (1H, m); 2.26 (1H, s); 2.47 (2H, m); 2.56 (1H, dd, J=5.3, 13.4); 2.83 (1H, dd, J=7.2, 9.6); 3.76 (3H, s); 5.36 (1H, dd, J=9.9, 9.9); 5.53 (1H, dd, J=5.4, 11.7); 6.40 (1H, dd, J=1.2, 1.8); 7.15 (1H, dd, J=3.8, 5.1); 7.42 (2H, m); 7.63 (1H, dd, J=1.3, 5.1); 7.89 (1H, dd, J=1.3, 3.8); ¹³C NMR (CDCl₃): δ 15.4, 16.7, 18.4, 31.1, 35.7, 38.4, 42.4, 43.6, 51.7, 52.2, 53.9, 64.3, 72.3, 75.7, 108.6, 125.4, 128.1, 132.6, 133.5, 134.5, 139.7, 143.9, 161.3, 171.3, 171.8, 201.8. anal. C, 62.66%; H, 5.84%; O, 25.67%; calcd for C₂₆H₂₈O₈S, C, 62.39%; H, 5.64%; O, 25.57%.

### Example 7

(2S,4aS,6aR,7R,9S,10aS,10bR)-9-(4-Methoxybenzoyloxy)-2-(3-furanyl)-dodecahydro-6a,10b-dimethyl-4,10-dioxo-2H-naphtho[2,1-c]pyran-7-carboxylic acid methyl ester (10e); 1.17 (3H, s); 1.46 (3H, s); 1.63 (3H, m); 1.83 (1H, dd, J=3.0, 9.0); 2.10 (1H, dd, J=9.0, 11.7); 2.17 (1H, m); 2.26 (1H, s); 2.45 (2H, m); 2.54 (1H, dd, J=5.1, 13.8); 2.83 (1H, dd, J=7.5, 9.6); 3.74 (3H, s); 3.87 (3H, s); 5.37 (1H, dd, J=9.3, 10.8); 5.51 (1H, dd, J=5.1, 11.7); 6.38 (1H, dd, J=0.9, 1.8); 6.93 (2H, dt, J=2.1, 2.7, 9.0); 7.39 (1H, dd, J=1.5, 1.8); 7.41 (1H, dd, J=0.9, 1.5); 8.04 (1H, dt, J=2.1, 2.7, 9.0)

### Example 8

(2S,4aS,6aR,7R,9S,10aS,10bR)-9-(4-Nitrobenzoyloxy)-2-(3-furanyl)-dodecahydro-6a,10b-dimethyl-4,10-dioxo-2H-naphtho[2,1-c]pyran-7-carboxylic acid methyl ester (10f); 1.18 (3H, s); 1.46 (3H, s); 1.64 (3H, m); 1.84 (2H, dd, J=2.7, 9.9); 2.11 (1H, dd, J=3.0, 10.8); 2.27 (1H, s); 2.51 (3H, m); 2.85 (1H, dd, J=7.2, 9.6); 3.76 (3H, s); 5.42 (1H, dd, J=9.6, 10.5); 5.53 (1H, dd, J=5.4, 11.7); 6.39 (1H, dd, J=0.9, 1.5); 7.40 (1H, dd, J=1.5, 1.8); 7.42 (1H, dd, J=0.6, 1.5); 8.29 (4H, m)

### Example 9

Thiophene-3-carboxylic acid (2S,4aR,6aR,7R,9S,10aS,10bR)-7-carbomethoxy-2-(3-furanyl)dodecahydro-6a,10b-dimethyl-4,10-dioxo-2H-naphtho[2,1-c]pyran-9-yl ester (10g). 10g was synthesized from 7 using EDCI coupling to afford a white solid, mp 211-212° C.; ¹H NMR (CDCl₃): δ 1.18 (3H, s); 1.48 (3H, s); 1.54-1.77 (3H, m); 1.84 (1H, dd, J=3.0, 10.2); 2.12 (1H, dd, J=2.2, 11.1); 2.19 (1H, m); 2.26 (1H, s); 2.47 (2H, m); 2.56 (1H, dd, J=5.3, 13.4); 2.83 (1H, dd, J=7.2, 9.6); 3.76 (3H, s); 5.36 (1H, dd, J=9.9, 9.9); 5.53 (1H, dd, J=5.4, 11.7); 6.40 (1H, s); 7.27 (1H, s); 7.34 (2H, dd, J=3.0, 5.1); 7.40-7.42 (2H, m); 7.56-7.57 (1H, m); 8.21-8.22 (1H, m); anal. C, 62.55%; H, 5.66%; O, 25.73%; calcd for C₂₆H₂₈O₈S, C, 62.39%; H, 5.64%; O, 25.57%.

### Example 10

Benzofuran-2-carboxylic acid (2S,4aR,6aR,7R,9S,10aS,10bR)-7-carbomethoxy-2-(3-furanyl)dodecahydro-6a,10b-dimethyl-4,10-dioxo-2H-naphtho[2,1-c]pyran-9-yl ester (10h). 10h was synthesized from 7 using EDCI coupling to afford a white solid, mp 226-227° C.; ¹H NMR (CDCl₃): δ 1.18 (3H, s); 1.48 (3H, s); 1.54-1.77 (3H, m); 1.84 (1H, dd, J=3.0, 10.2); 2.12 (1H, dd, J=2.2, 11.1); 2.19 (1H, m); 2.26 (1H, s); 2.47 (2H, m); 2.56 (1H, dd, J=5.3, 13.4); 2.83 (1H, dd, J=7.2, 9.6); 3.76 (3H, s); 5.36 (1H, dd, J=9.9, 9.9); 5.53 (1H, dd, J=5.4, 11.7); 6.40 (1H, m); 7.31-7.36 (1H, m); 7.41-7.43 (2H, m); 7.46-7.51 (1H, m); 7.60-7.66 (2H, m); 7.70-7.73 (1H, m); anal. C, 67.24%; H, 5.65%; O, 27.20%; calcd for C₃₀H₃₀O₉, C, 67.41%; H, 5.66%; O, 26.94%.

### Example 11

(2S,4aS,6aR,7R,9S,10aS,10bR)-9-(Benzenesulfonyloxy)-2-(3-furanyl)-dodecahydro-6a,10b-dimethyl-4,10-dioxo-2H-naphtho[2,1-c]pyran-7-carboxylic acid methyl ester (11a). A solution of 7 (100 mg, 0.26 mmol), benzenesulfonyl chloride (66 μL, 0.51 mmol), pyridine (0.23 mL, 2.74 mmol) and a catalytic amount of DMAP in CH₂Cl₂ (30 mL) was stirred at room temperature overnight. The mixture was diluted with CH₂Cl₂ (40 mL) and washed sequentially with 2N HCl (2×30 mL), saturated NaHCO₃ (2×30 mL) and water (40 mL). The organic extract was dried (Na₂SO₄), filtered and concentrated to an oil which was purified by flash column chromatography (40% ethyl acetate/hexanes) to give 103 mg (76%) of 11a as a white solid, mp 157-159° C. (dec); ¹H NMR (CDCl₃): δ 1.10 (3H, s); 1.44 (3H, s); 1.47-1.71 (3H, m); 1.82 (1H, m); 2.06 (1H, dd, J=3.0, 12.0), 2.10 (1H, s); 2.19 (1H, m); 2.26-2.46 (3H, m); 2.72 (1H, dd, J=4.2, 12.3); 3.73 (3H, s); 5.00 (1H, dd, J=8.1, 11.7); 5.53 (1H, dd, J=5.0, 11.6); 6.40 (1H, m); 7.44 (2H, m); 7.58 (2H, m); 7.67 (1H, m). 8.01 (1H, m); ¹³C NMR (CDCl₃): δ 15.3, 16.6, 18.3, 32.4, 35.7, 38.3, 42.2, 435.5, 51.5, 52.3, 53.7, 64.6, 72.1, 80.0, 108.6, 125.4, 126.6, 128.0, 128.1, 129.4 (2), 134.2, 139.6, 144.0, 171.1, 171.2, 200.0. anal. C, 61.16%; H, 5.85%; O, 27.06%; calcd for C₂₇H₃₀O₉S, C, 61.62%; H, 5.70%; O, 27.14%.

### Example 12

(2S,4aS,6aR,7R,9S,10aS,10bR)-9-(4-Methylbenzenesulfonyloxy)-2-(3-furanyl)-dodecahydro-6a,10b-dimethyl-4,10-dioxo-2H-naphtho[2,1-c]pyran-7-carboxylic acid methyl ester (11b). 11b was synthesized as described for 11a from 7 using 4-methylbenzenesulfonyl chloride to afforded 10 mg (0.22 mmol, 86%) of 11b as a white solid, mp 163-165° C. (dec); ¹H NMR (CDCl₃): δ 1.10 (3H, s); 1.44 (3H, s); 1.47-1.67 (5H, m); 1.81 (1H, dd, J=2.7, 9.9); 2.05-2.08 (1H, dd, obscured); 2.08 (1H, s); 2.17 (1H, dd, J=3.0, 10.2); 2.32-2.44 (3H, m); 2.41 (3H, s); 2.72 (1H, dd, J=4.2, 12.3); 3.73 (3H, s); 4.96 (1H, dd, J=7.5, 11.7); 5.53 (1H, dd, J=5.1, 11.7); 6.40 (1H, dd, J=1.2, 1.2); 7.34 (2H, d, J=7.8); 7.43 (2H, m); 7.84 (2H, d, J=7.8). ¹³C NMR (CDCl₃): δ 15.3, 16.6, 18.3, 21.8, 32.5, 35.7, 38.3, 42.2, 43.5, 51.6, 52.3, 53.7, 64.6, 72.1, 77.4, 79.8, 108.6, 125.4, 128.1, 130.0, 139.7, 144.0, 145.4, 171.1, 171.2, 200.1. anal. C, 61.17%; H, 5.84%; O, 27.07%; calcd for C₂₈H₃₂O₉S.0.25H₂O, C, 61.24%; H, 5.97%; O, 26.95%.

### Example 13

(2S,4aS,6aR,7R,9S,10aS,10bR)-9-(4-Methoxybenzenesulfonyloxy)-2-(3-furanyl)-dodecahydro-6a,10b-dimethyl-4,10-dioxo-2H-naphtho[2,1-c]pyran-7-carboxylic acid methyl ester (11c). 11c was synthesized as described for 11a from 7 using 4-methoxybenzenesulfonyl chloride to afforded a white solid, mp 148-149° C. (dec); ¹H NMR (CDCl₃): δ 1.10 (3H, s); 1.44 (3H, s); 1.47-1.67 (5H, m); 1.81 (1H, dd, J=2.7, 9.9); 2.05-2.08 (1H, dd, obscured); 2.08 (1H, s); 2.17 (1H, dd, J=3.0, 10.2); 2.32-2.44 (3H, m); 2.41 (3H, s); 2.72 (1H, dd, J=4.2, 12.3); 3.73 (3H, s); 4.96 (1H, dd, J=7.5, 11.7); 5.53 (1H, dd, J=5.1, 11.7); 6.40 (1H, s); 6.98-7.04 (3H, m); 7.43 (2H, s); 7.86-7.91 (3H, m); anal. C, 59.48%; H, 5.79%; O, 28.97%; calcd for C₂₈H₃₂O₁₀S, C, 59.33%; H, 5.53%; O, 29.27%.

### Example 14

(2S,4aS,6aR,7R,9S,10aS,10bR)-9-(4-Bromobenzenesulfonyloxy)-2-(3-furanyl)-dodecahydro-6a,10b-dimethyl-4,10-dioxo-2H-naphtho[2,1-c]pyran-7-carboxylic acid methyl ester (11d). 11d was synthesized as described for 11a from 7 using 4-bromobenzenesulfonyl chloride to afforded a white solid, mp 165-166° C.; ¹H NMR (CDCl₃): δ 1.10 (3H, s); 1.44 (3H, s); 1.47-1.67 (5H, m); 1.81 (1H, dd, J=2.7, 9.9); 2.05-2.08 (1H, dd, obscured); 2.08 (1H, s); 2.17 (1H, dd, J=3.0, 10.2); 2.32-2.44 (3H, m); 2.41 (3H, s); 2.72 (1H, dd, J=4.2, 12.3); 3.73 (3H, s); 4.96 (1H, dd, J=7.5, 11.7); 5.53 (1H, dd, J=5.1, 11.7); 6.39-6.40 (1H, m); 7.43-7.44 (2H, s); 7.68-7.72 (2H, dt, J=1.8, 2.1, 8.4); 7.81-7.85 (2H, dt, J=2.4, 1.8, 8.4); anal. C, 53.11%; H, 4.94%; O, 23.41%; calcd for C₂₈H₃₂O₁₀S, C, 53.21%; H, 4.80%; O, 23.63%.

### Example 15

(2S,4aS,6aR,7R,9S,10aS,10bR)-9-(2-Bromobenzenesulfonyloxy)-2-(3-furanyl)-dodecahydro-6a,10b-dimethyl-4,10-dioxo-2H-naphtho[2,1-c]pyran-7-carboxylic acid methyl ester (11e). 11e was synthesized as described for 11a from 7 using 2-bromobenzenesulfonyl chloride to afforded a white solid, mp 162-163° C.; ¹H NMR (CDCl₃): δ 1.10 (3H, s); 1.44 (3H, s); 1.47-1.67 (5H, m); 1.81 (1H, dd, J=2.7, 9.9); 2.05-2.08 (1H, dd, obscured); 2.08 (1H, s); 2.17 (1H, dd, J=3.0, 10.2); 2.32-2.44 (3H, m); 2.41 (3H, s); 2.72 (1H, dd, J=4.2, 12.3); 3.73 (3H, s); 4.96 (1H, dd, J=7.5, 11.7); 5.53 (1H, dd, J=5.1, 11.7); 6.40 (1H, s); 7.43 (2H, s); 7.48-7.50 (1H, t, J=3.6); 7.77-7.80 (1H, m); 8.15-8.18 (1H, m); anal. C, 52.73%; H, 4.84%; O, 23.26%; calcd for C₂₈H₃₂O₁₀S, C, 53.21%; H, 4.80%; O, 23.63%.

### Example 16

(2S,4aR,6a,7R,9S,10aS,10bR)-9-(Benzoylamino)-2-(3-furanyl)-dodecahydro-6a,10b-dimethyl-4,10-dioxo-2H-naphtho[2,1-c]pyran-7-carboxylic acid methyl ester (13a). A solution of 12 (0.10 g, 0.26 mmol), benzoyl chloride (0.11 g, 0.78 mmol) and DMAP (0.08 g, 0.78 mmol) in CH₂Cl₂ (20 mL) was stirred at room temperature for 2 h. Absolute MeOH (15 mL) was added and the solvent was removed under reduced pressure. CH₂Cl₂ (25 mL) was added to the residue and the solution was washed with 10% HCl (2×20 mL), H₂O (3×20 mL), and saturated NaCl (3×20 mL) and dried (Na₂SO₄). Removal of the solvent under reduced pressure afforded 0.09 g (67%) of 13a as a white crystalline solid, mp 155-157° C. (EtOAc/n-hexanes); ¹H NMR (CDCl₃): δ 1.44 (3H, s); 1.50 (3H, s); 1.63 (3H, m); 1.82 (1H, dd, J=2.1, 10.5); 2.0 (1H, m); 2.12 (1H, dd, J=2.7, 8.4); 2.17 (1H, m); 2.32 (1H, s); 2.48 (1H, dd, J=5.4, 13.2); 2.79 (1H, dd, J=3.3, 6.9); 2.87 (1H, dd, J=2.7, 13.5); 3.71 (3H, s); 4.69 (1H, m); 5.55 (1H, dd, J=5.1, 11.4); 6.37 (1H, dd, J=0.9, 1.8); 7.1 (1H, d, J=6.0); 7.39 (1H, t, J=1.8); 7.41 (1H, dd, J=0.9, 1.8); 7.46 (1H, m); 7.53 (1H, tt, J==1.5, 2.7, 7.2); 7.80 (1H, t, J=2.4); 7.82 (1H, t, J=1.2); Anal. (C₂₈H₃₁NO₇.0.5H₂O): C, H, N.

### Example 17

(2S,4aR,6aR,7R,9S,10aS,10bR)-9-(4-Bromobenzoylamino)-2-(3-furanyl)-dodecahydro-6a,10b-dimethyl-4,10-dioxo-2H-naphtho[2,1-c]pyran-7-carboxylic acid methyl ester. 13b was synthesized as described for 13a from 12 using 4-bromobenzoyl chloride; ¹H NMR (CDCl₃): δ 1.17 (3H, s); 1.46 (3H, s); 1.52-1.69 (3H, m); 1.83 (1H, dd, J=3.0, 9.9); 2.10 (1H, dd, J=2.7, 11.4); 2.21 (1H, m); 2.25 (1H, s); 2.43-2.57 (3H, m); 2.83 (1H, dd, J=8.1); 3.75 (3H, s); 5.38 (1H, dd, J=9.9, 9.9), 5.52 (1H, dd, J=5.4, 12.0); 6.39 (1H, dd, J=0.9, 1.8); 7.40 (1H, dd, J=1.8, 1.8); 7.42 (1H, dd, J=0.9, 1.8); 7.61 (2H, m); 7.95 (2H, m). ¹³C NMR (CDCl₃): δ 15.4, 16.7, 18.4, 31.1, 35.7, 38.4, 42.4, 43.7, 51.6, 52.3, 53.9, 64.4, 72.3, 75.9, 108.6, 125.4, 128.2, 129.0, 131.6, 132.0, 139.7, 143.9, 165.0, 171.3, 171.7, 201.9

### Example 18

(2S,4aR,6aR,7R,9S,10aS,10bR)-9-(4-Nitrobenzoylamino)-2-(3-furanyl)-dodecahydro-6a,10b-dimethyl-4,10-dioxo-2H-naphtho[2,1-c]pyran-7-carboxylic acid methyl ester (13c). 13c was synthesized as described for 13a from 12 using 4-nitrobenzoyl chloride; ¹H NMR (CDCl₃): δ 1.18 (3H, s); 1.46 (3H, s); 1.58-1.70 (3H, m); 1.84 (1H, dd, J=3.0, 9.6); 2.11 (1H, dd, J=2.7, 12.6); 2.19 (1H, m); 2.27 (1H, s); 2.46-2.55 (3H, m); 2.85 (1H, dd, J=6.0, 10.8); 3.76 (3H, s); 5.42 (1H, dd, J=9.3, 10.8), 5.53 (1H, dd, J=5.1, 11.7); 6.39 (1H, dd, J=0.9, 1.8); 7.40 (1H, dd, J=1.9, 3.7); 7.42 (1H, dd, J=0.9, 1.8); 8.25 (2H, dt, J=1.8, 2.1, 8.7); 7.95 (2H, dt, J=1.8, 2.1, 9.3).

### Example 19

(2S,4aR,6aR,7R,9S,10aS,10bR)-9-(Phenylcarbamoyloxy)-2-(3-furanyl)-dodecahydro-6a,10b-dimethyl-4,10-dioxo-2H-naphtho[2,1-c]pyran-7-carboxylic acid methyl ester (14a). A solution of 7 (0.06 g, 0.14 mmol), phenylisocyanate (0.1 mL, 0.28 mmol) in CHCl₃ was heated at reflux overnight. MeOH was added to the solution and the solvent was removed under reduced pressure. CH₂Cl₂ was added and the solution was then washed with 5% NaHCO₃ (10 mL), 2N HCl (10 mL), saturated NaCl, and dried (Na₂SO₄). The solvent was removed under reduced pressure to give a crude oil. The oil was purified by column chromatography (eluent: EtOAc/n-hexanes) to afford 0.07 g (93%) of 14a as a clear oil: ¹H NMR (CDCl₃): δ 1.15 (3H, s, H-19); 1.48 (3H, s, H-20); 1.52-1.72 (3H, m, H-7α,β and H-11β); 1.82 (1H, ddd, J=2.7, 2.7, 10.2 Hz, H-6α); 2.03-2.20 (1H, m, H-8); 2.22 (1H, s, H-10); 2.24-2.48 (2H, m, H-3α,β); 2.54 (1H, dd, J=5.1, 13.5 Hz, H-4); 2.80 (1H, dd, J=3.6, 13.2 Hz, H-11α); 3.75 (3H, s, CO₂CH₃); 5.21 (1H, dd, J=7.8, 12.3 Hz, 11-2); 5.54 (1H, dd, J=5.7, 11.7 Hz, H-12); 6.40 (1H, m, H-14); 6.69 (1H, br s, NH); 6.87 (1H, br s, Ar—H); 7.12 (2H, m, Ar—H's); 7.28-7.45 (5H, m, H-14, H-15 and Ar—H's); Anal. (C₂₈H₃₁NO₈. 1.25H₂O): C, H, N.

### Example 20

(2S,4aR,6aR,7R,9S,10aS,10bR)-9-(4-Bromophenylcarbamoyloxy)-2-(3-furanyl)-dodecahydro-6a,10b-dimethyl-4,10-dioxo-2H-naphtho[2,1-c]pyran-7-carboxylic acid methyl ester (14b). 14b was synthesized as described for 14a from 12 using 4-bromophenylisocynate; ¹H NMR (CDCl₃): δ 1.15 (3H, s, H-19); 1.48 (3H, s, H-20); 1.52-1.72 (3H, m, H-7α,β and H-11β); 1.82 (1H, ddd, J=2.7, 2.7, 10.2 Hz, H-6α); 2.03-2.20 (1H, m, H-8); 2.22 (1H, s, H-10); 2.24-2.48 (2H, m, H-3α,β); 2.54 (1H, dd, J=5.1, 13.5 Hz, H-4); 2.80 (1H, dd, J=3.6, 13.2 Hz, H-11α); 3.75 (3H, s, CO₂CH₃); 5.21 (1H, dd, J=7.8, 12.3 Hz, H-2); 5.54 (1H, dd, J=5.7, 11.7 Hz, H-12); 6.56 (1H, m, H-14); 7.55 (1H, m, Ar—U); 7.63 (1H, m, Ar—H's); 7.78-7.83 (2H, m, H-14, H-15 and Ar—H's); 8.20-8.25 (2H, m, Ar—H).

### Example 21

(2S,4aR,6aR,7R,9S,10aS,10bR)-9-(4-Nitrophenylcarbamoyloxy)-2-(3-furanyl)-dodecahydro-6a,10b-dimethyl-4,10-dioxo-2H-naphtho[2,1-c]pyran-7-carboxylic acid methyl ester (14c). 14c was synthesized as described for 14a from 12 using 4-nitrophenylisocynate, mp: 251-253° C.; ¹H NMR (CDCl₃): δ 1.15 (3H, s, H-19); 1.48 (3H, s, H-20); 1.52-1.72 (3H, m, H-7α,β and H-11β); 1.82 (1H, ddd, J=2.7, 2.7, 10.2 Hz, H-6 oz); 2.03-2.20 (1H, m, H-8); 2.22 (1H, s, H-10); 2.24-2.48 (2H, m, H-3α,β); 2.54 (1H, dd, J=5.1, 13.5 Hz, H-4); 2.80 (1H, dd, J=3.6, 13.2 Hz, H-11α); 3.75 (3H, s, CO₂CH₃); 5.21 (1H, dd, J=7.8, 12.3 Hz, H-2); 5.54 (1H, dd, J=5.7, 11.7 Hz, H-12); 6.56 (1H, m, H-14); 7.55 (1H, m, Ar—H); 7.63 (1H, m, Ar—H's); 7.78-7.83 (2H, m, H-14, H-15 and Ar—H's); 8.20-8.25 (2H, m, Ar—H).

### Example 22

(2S,4aS,6aR,7R,9S,10aS,10bR)-9-(4-Bromobenzoyloxy)-2-(3-furanyl)-dodecahydro-6a,10b-dimethyl-4,10-dioxo-2H-naphtho[2,1-c]pyran-7-carboxylic acid methyl ester (10c). 10c was synthesized as described for 8a from 7 using 4-bromobenzoyl chloride to afford the title compound.

### Example 23

### Biological activity of Compound Vb (Herkinorin)

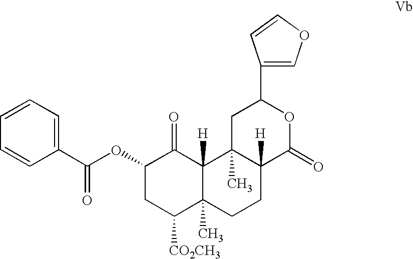

### Methods:

Drugs: DAMGO, morphine sulfate, naloxone and nor-binaltorphimine (nor-BNI) were purchased from commercial suppliers. DAMGO (Tocris, place) and morphine sulfate (Sigma, St. Louis, Mo.) were prepared as 10 mM stocks in phosphate buffered saline. Compound Vb was prepared in DMSO for a 10 mM stock. Dilutions were made into minimal essential media prior to treating cells. Compound Vb was prepared in a vehicle of either EtOH/Cremophor/H₂O, 1:1:8 or DMSO/Cremophor/H₂O, 1:1:8 for injection into animals. Naloxone (Sigma, St. Louis, Mo.) and nor-BNI (Sigma, St. Louis, Mo.) were dissolved in 0.9% NaCl.

Animals: Male Sprague-Dawley rats (Harlan, Indianapolis, Ind.) weighing 250-275 g were used. Rats were housed one to two per cage with free access to food and water, and maintained on a 12-h light/dark cycle in the Association for the Assessment and Accreditation of Laboratory Animal Care-approved animal care facility. The Institutional Animal Care and Use Committee of The University of Iowa approved all experimental procedures.

Phospho-ERK1/2 immunoblot assay: Phospho-ERK1/2 immunoblot assay: HEK-293 stably expressing an hemagglutinin (HA-N-terminus) tagged mouse μOR (˜2 pmol/mg membrane protein) were assessed for agonist induced ERK1/2 phosphorylation. Cells were serum starved at 37° C. under 5% CO₂ for 30 min prior to drug treatment. After 10 min of drug treatment, cell lysates were prepared on ice in lysing buffer (20 mM Tris HCl pH 7.5, 150 mM NaCl, 2 mM EDTA, 0.1% SDS, 1% NP40, 0.25% deoxycholate, 1 mM sodium orthovanadate, 1 mM PMSF, 1 mM NaF, with Complete Mini, EDTA-free protease inhibitor cocktail tablet (Roche Diagnostics, Indianapolis, Ind.)). Protein levels were determined by the Bio-Rad DC protein assay system (BioRad, Hercules, Calif.) and 20 μg protein per lane were resolved by 1-D gel electrophoresis on 10% Bis-Tris gels (BioRad or Invitrogen, Carlsbad, Calif.). Proteins were then transferred to polyvinylidene fluoride (PVDF) membranes (Immobilon P, Millipore, Billerica, Mass.) and immunoblotted for phospho ERK1/2 (p-ERK E-4: sc-7383, Santa Cruz Biotechnology, Inc., Santa Cruz, Calif.). Blots were then stripped and blotted for total ERK1/2 levels (p44/42 MAP Kinase Antibody, Cell Signaling Technology, Danvers, Mass.) which were used to normalize the overall phosphorylation of ERK1/2 between samples (Bohn, L. M., Belcheva, M. M. & Coscia, C. J., 2000, J Neurochem 74, 564-73). Chemiluminescence was detected and quantified using the Kodak 2000R imaging system (Eastman Kodak Company, Rochester, N.Y.) and GraphPad Prism software (GraphPad Software, Inc., San Diego, Calif.).

Cellular Trafficking. HEK-293 cells were transiently transfected with combinations of the following cDNA as indicated in the figure legends: hemagglutinin (HA-N-terminus) tagged mouse FOR (10 μg cDNA); beta-arrestin2 tagged with green fluorescent protein (βarr2-GFP) (2 μg cDNA); mouse FOR tagged at the C-terminus with yellow fluorescence protein (μOR-YFP); and GRK2 (5 μg cDNA). In some cases, cells stably expressing the FOR were used and no differences were observed when compared to transiently transfected cells. Cell media was changed 10-20 minutes prior to addition of drug to serum-free MEM. Cells were visualized using an Olympus confocal microscope with Green-Helium Neon and Argon Lasers. Multiple cells were recorded per dish following more than four separate transfection experiments; shown are representative cells. Transfections and imaging were performed as previously described (Bohn, L. M., Dykstra, L. A., Lefkowitz, R. J., Caron, M. G. & Barak, L. S., 2004, Mol Pharmacol 66, 106-12).

Immunoprecipitation: HEK-293 cells stably expressing HA-tagged gμOR (˜2 pmol/mg protein) were grown to 75% confluence at 5% CO₂ and 37° C. in MEM. Cells were serum starved for 15 minutes at 5% CO₂ and 37° C. and then treated for 10 minutes at 5% CO₂ and 37° C. with saline, DAMGO (1 μM), Morphine (10 μM), or Compound Vb (10 μM). As a control, mock transfected cells were treated with DAMGO (1 μM). Cells were placed on ice and the media containing drug was aspirated, followed by washing with 3 mL cold PBS. Cells were lysed with 250 μl Ripa-P+ buffer (20 mM Tris HCl pH8.0, 150 mM NaCl, 2 mM EDTA, 0.1% SDS, 1% NP-40, 0.25% deoxycholate, 1 mM sodium orthovanadate, 1 mM PMSF, 1 mM NaF, and protease inhibitor cocktail (Roche Diagnostics, Indianapolis, Ind.). Cell lysates were collected and solubilized at 4° C. for 1 hour. The insoluble fraction was removed with a 20,000×g spin at 4° C. for 30 minutes. Protein content was measured using Bio-Rad D_{c} Protein Assay and samples were diluted to equal concentrations. Equal amounts of protein (700-1000 μg) or buffer only (for “no protein” control) were incubated with the 70 μl of a 1:1 suspension of monoclonal anti-HA-agarose beads (Sigma, St. Louis, Mo.) overnight (˜16 hours) at 4° C. with rotation. The immunoprecipitate complex was collected and washed per manufacturer's instructions. Proteins were eluted from anti-HA-agarose in 30 μl 4×XT Sample Buffer (BioRad, Hercules, Calif.) (62.5 mM Tris-HCl, pH6.8, 25% glycerol, 2% SDS, 0.01% bromophenol blue) with 5% β-mercaptoethanol at 95° C. for 4 minutes. Samples were resolved on 10% Bis-Tris XT Precast Gels (Bio-Rad) and proteins were transferred to PVDF membranes (Immobilon-P, Millipore, Billerica, Mass.). Membranes were incubated with a phospho-μOR antibody (1:500) that recognizes phosphorylated serine 375 of the mouse μOR (p-μOR Ser375, Cell Signaling, Danvers, Mass.). Chemiluminescence was visualized using a Kodak 2000R image station. Membranes were stripped and blotted with a primary antibody against the C-terminus of the μOR (1:1000) (Sigma, St. Louis, Mo.) to determine total levels of receptor per lane. Densitometry was assessed using the Kodak imaging software and p-μOR levels were normalized to the total receptor per lane as well as to the degree of stimulation compared to saline-treated controls in each blot. Data were analyzed using GraphPad Prism software (GraphPad Software, Inc., San Diego, Calif.).

Immunofluorescence of Cell Surface Receptors: Stably transfected HA-N-terminus tagged μOR-expressing HEK-293 cells were plated on a 96 well optical plate (collagen coated) and treated with either DAMGO or Compound Vb for the times indicated. Cells were washed 2× with MEM, fixed with 4% PFA room temp for 20 min and blocked in MEM+goat serum for 1 hour. Cells were then incubated with an anti-HA antibody (monoclonal clone 12CA5, Roche Diagnostics, Indianapolis, Ind.) 1:500 in blocking buffer overnight at 4° C. Cells were washed 2× in blocking buffer and incubated for 1 h with the Alexafluor-488 goat anti-mouse secondary antibody (Molecular Probes) at room temperature. Following 2 washes in phosphate buffered saline, immunofluorescence was assessed using a Fusion Plate Reader (Perkin Elmer, Boston, Mass.). Samples that were treated under the same conditions were examined under the confocal microscope to assure that only cell surface labeling was assessed (data not shown). Data are normalized to the control in which no agonists were added. Non-specific secondary antibody interactions were subtracted from each point.

Formalin Rat Paw Withdrawal Test: Rats were placed in clear plexiglass testing chambers (30.5×30.5×30.5 cm) and allowed to acclimate for 20 minutes. A mirror was placed at a 45° angle below the floor to allow for an unobstructed view of the rats paws. No restraint was placed on the rats and a HEPA filter was continuously run in order to mask external noise. Following the 20-min acclimation period, animals received a 100 μL injection of either Compound Vb (˜0.04 mg/kg) or vehicle subcutaneously in the paw and were placed back into the testing chamber for 5 minutes (Joshi et al., 2000; Joshi and Gebhart, 2003). Animals were then injected with 100 μL of a 1.25% formalin solution into the plantar surface according to the methods previously described (Dubuisson, D. & Dennis, S. G., 1997, Pain 4, 161-74; Wheeler-Aceto, H. & Cowan, A., 1991, Psychopharmacology (Berl) 104, 35-44; and Kaneko, M. & Hammond, D. L., 1997, J Pharmacol Exp Ther 282, 928-38). Behavior was observed and tracked for 1 hour and the number of flinches was recorded using a Windows XP-based (Microsoft, Seattle, Wash.) program. Each drug treatment group consisted of 6 rats and no rat was tested more than once. For the antagonist studies, rats were pretreated with either nor-binaltorphimine (nor-BNI) (5 mg/kg, 24 hours prior to agonist) or naloxone (5 mg/kg s.c. 30 min prior to agonist) subcutaneously behind the neck.

Chronic study: Chronic animals were given vehicle or Compound Vb alternating dorsal and plantar surface to avoid sensitization caused from injections at the same site over a 5 day period. Following administration of drug on the fifth day, rats were then administered formalin and observed as described above.

Statistics: Statistical analyses were performed using the GraphPad Prism software (GraphPad Software, Inc., San Diego, Calif.) and the specific tests used are presented in the figure legends.

### Results:

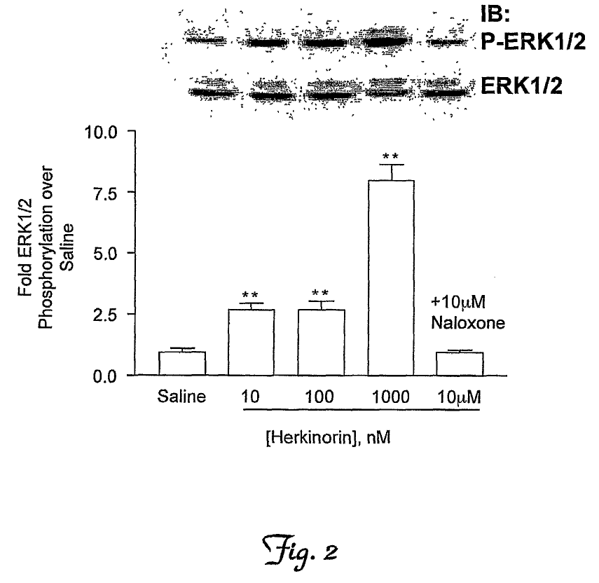

The chemical synthesis of Compound Vb from salvinorin A has been previously described (Harding, W. W., Tidgewell, K., Byrd, N., Cobb, H., Dersch, C. M., Butelman, E. R., Rothman, R. B. & Prisinzano, T. E., 2005, J Med Chem 48, 4765-71). Ligand affinity and agonist activity were determined at the μ, δ and κ opioid receptors in CHO cells stably expressing each receptor type and Compound Vb was reported to have a greater affinity for μOR over κOR and no affinity for the δOR. Like other FOR agonists, such as the enkephalin analog, [D-Ala², N-MePhe⁴, Gly⁵-ol]Enkephalin (DAMGO), Compound Vb dose dependently activates the Map Kinases, ERK1/2, in a μOR antagonist reversible manner see FIG. 2; Belcheva et al., 1998). Similar results were also obtained using CHO cells expressing the human μOR.

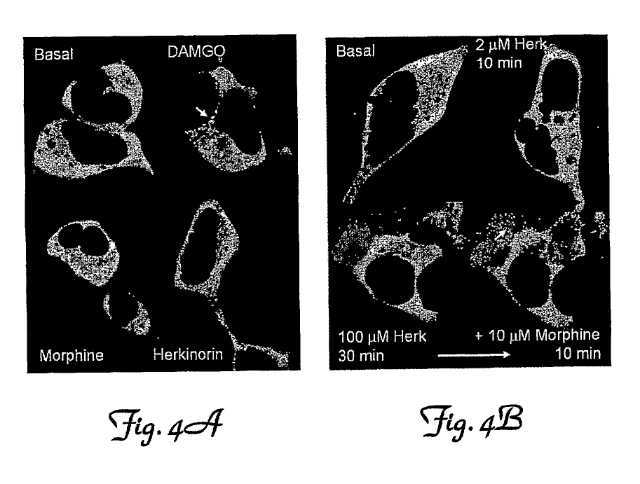

Since agonist activation of most GPCRs leads to GRK-mediated receptor phosphorylation, β-arrestin recruitment and internalization, Compound Vb was compared to other opiate agonists for their ability to regulate the μOR in HEK-293 cells, a model system routinely used to study opioid receptor regulation and trafficking. While both morphine and DAMGO activate the μOR, morphine is much less effective in promoting receptor phosphorylation (FIG. 3), β-arrestin recruitment (FIG. 4A), and μOR internalization (FIG. 5A) (Zhang, J., Ferguson, S. S., Barak, L. S., Bodduluri, S. R., Laporte, S. A., Law, P. Y. & Caron, M. G., 1998, Proc Natl Acad Sci USA 95, 7157-62; Whistler, J. L. & von Zastrow, M., 1998, Proc Natl Acad Sci USA 95, 9914-9; Bohn, L. M., Dykstra, L. A., Lefkowitz, R. J., Caron, M. G. & Barak, L. S., 2004, Mol Pharmacol 66, 106-12; Bailey, C. P., Couch, D., Johnson, E., Griffiths, K., Kelly, E. & Henderson, G., 2003, J Neurosci 23, 10515-20; Schulz, S., Mayer, D., Pfeiffer, M., Stumm, R., Koch, T. & Hollt, V., 2004, Embo J23, 3282-9; and Koch, T., Widera, A., Bartzsch, K., Schulz, S., Brandenburg, L. O., Wundrack, N., Beyer, A., Grecksch, G. & Hollt, V., 2005, Mol Pharmacol 67, 280-7). Like morphine, Compound Vb does not promote robust phosphorylation of the μOR at serine-375 (FIG. 3). Compound Vb also resembles morphine in that it does not lead to the robust recruitment βarr2-GFP (FIG. 4A) nor does it promote μOR internalization (FIG. 5A) as does DAMGO treatment. The lack of Compound Vb-induced receptor internalization is further demonstrated by immunolabeling the N-terminus of receptors in intact, nonpermeabilized HEK-293 cells following drug treatment over 2 hours. While DAMGO treated cells show a loss of 40% of cell surface receptors, the Compound Vb treated cells maintain the same level of cell surface receptor expression over the 2 hour time period (FIG. 5B).

The overexpression of GRKs permits morphine-induced βarr2-GFP translocation (FIG. 4B) as well as FOR internalization (FIG. 5C), suggesting that the agonist occupancy promotes a conformation that differs between DAMGO and morphine at the level of GRK-mediated phosphorylation of the morphine-bound receptor (Zhang, J., Ferguson, S. S., Barak, L. S., Bodduluri, S. R., Laporte, S. A., Law, P. Y. & Caron, M. G., 1998, Proc Natl Acad Sci USA 95, 7157-62; and Bohn, L. M., Dykstra, L. A., Lefkowitz, R. J., Caron, M. G. & Barak, L. S., 2004, Mol Pharmacol 66, 106-12). However, overexpression of GRK2 is insufficient to promote Compound Vb-induced βarr2-GFP translocation (FIG. 4B) or μOR internalization (FIG. 5C) further demonstrating the differences between morphine and Compound Vb in respect to agonist-induced FOR trafficking.

Another means by which GPCR phosphorylation and internalization can be enhanced is by changing the serine and threonine numbers in the C-terminal tail (Oakley, R. H., Laporte, S. A., Holt, J. A., Barak, L. S. & Caron, M. G., 2001, J Biol Chem 276, 19452-60). A naturally occurring splice variant of the mouse μOR, μOR1-D, differs from the 10R only in the C-terminal sequence and has been shown to recruit βarr2-GFP and internalize following morphine treatment (Bohn, L M unpublished observations; Koch, T., Schulz, S., Pfeiffer, M., Klutzny, M., Schroder, H., Kahl, E. & Hollt, V., 2001, J Biol Chem 276, 31408-14; and Pan, Y. X., Xu, J., Bolan, E., Abbadie, C., Chang, A., Zuckerman, A., Rossi, G. & Pasternak, G. W., 1999, Mol Pharmacol 56, 396-403). The μOR1-D internalizes with morphine treatment while Compound Vb fails to internalize this receptor variant (FIG. 5D). Taken together, these findings demonstrate that unlike morphine, the Compound Vb-bound 10R does not internalize or recruit βarr2-GFP in HEK-293 cells and this cannot be overcome by overexpressing GRK2 or by substituting the μOR1-D splice variant.

To ascertain whether Compound Vb is active in vivo, the compound was tested for its ability to block nociceptive responses in the formalin-induced rat paw-withdrawal test which has been used to assess the peripheral analgesic effects of opioids (Dubuisson, D. & Dennis, S. G., 1997, Pain 4, 161-74; and Wheeler-Aceto, H. & Cowan, A., 1991, Psychopharmacology (Berl) 104, 35-44). Compound Vb produced significant antinociception in the second phase of the formalin paw-withdrawal test that was reversible by naloxone pretreatment indicating an effect at opioid receptors (FIG. 6A, Vehicle vs. Compound Vb: F_{(1,120)}=49.35, p<0.0001; Compound Vb vs. Compound Vb+Naloxone: F_{(1,120)}=4.24, p=0.0416, two-way ANOVA). Since naloxone is not selective for the μOR and since Compound Vb maintains some affinity at the μOR (Harding, W. W., Tidgewell, K., Byrd, N., Cobb, H., Dersch, C. M., Butelman, E. R., Rothman, R. B. & Prisinzano, T. E., 2005, J Med Chem 48, 4765-71), the κOR-selective antagonist nor-BNI was also tested against Compound Vb. Nor-BNI did not block Compound Vb's efficacy but rather significantly enhanced it (Compound Vb vs. Compound Vb+Nor-BNI: F_{(1,120)}=65.01, p<0.0001, two-way ANOVA) This is in agreement with studies that have shown that θOR activation can be nociceptive in the periphery (Pan, Z. Z., 1998, Trends Pharmacol Sci 19, 94-8). Therefore, it appears that Compound Vb's antinociceptive actions in vivo are primarily at the μOR.

Chronic opioid treatment, such as in regimens used for the long-term treatment of pain, leads to the development of opioid tolerance. Previously, a prominent role for βarr2 in the development of morphine tolerance in vivo has been shown (Bohn, L. M., Gainetdinov, R. R., Lin, F. T., Lefkowitz, R. J. & Caron, M. G., 2000, Nature 408, 720-3; and Bohn, L. M., Lefkowitz, R. J. & Caron, M. G., 2002, J Neurosci 22, 10494-500). Since Compound Vb does not lead to βarr2 recruitment in vitro (FIG. 4), rats were tested to determine if they would develop antinociceptive tolerance to Compound Vb in vivo. While morphine produces tolerance under similar testing conditions (Detweiler, D. J., Rohde, D. S. & Basbaum, A. I., 1995, Pain 63, 251-4; and Fazli-Tabaei, S., Yahyavi, S. H., Alagheband, P., Samie, H. R., Safari, S., Rastegar, F. & Zarrindast, M. R., 2005, Behav Pharmacol 16, 613-9), repeated administration of Compound Vb did not lead to the development of antinociceptive tolerance; interestingly, the chronic treatment led to significantly more Compound Vb-induced antinociception than seen on Day 1 (FIG. 6B, Vehicle Day 1 vs. Compound Vb Day 1: F_{(1,60)}=19.77, p<0.0001; Compound Vb Day 1 vs. Compound Vb Day 5: F_{(1,96)}=7.81, (p=0.0063); Vehicle Day 5 vs. Compound Vb Day 5: F_{(1,120)}=167.31, p<0.0001, two-way ANOVA). The most prominent effects of Compound Vb were on the second phase of the formalin test which is thought to reflect inflammatory contributions to nociception (Hunskaar, S. & Hole, K., 1987, Pain 30, 103-14). Analysis of the total number of flinches produced in this second phase upon acute and chronic administration of Compound Vb is presented in FIG. 6C. Compound Vb significantly inhibits formalin-induced flinching compared to vehicle in both treatment paradigms and this effect does not change following repeated dosing (Compound Vb vs. Vehicle, p<0.001; Compound Vb Day 1 vs. Compound Vb Day 5, p>0.05, one-way ANOVA analysis of variance; Bonferroni's multiple comparison test).

### Discussion:

Compound Vb is a potent opioid agonist that does not promote robust receptor phosphorylation (FIG. 2), βarr2-GFP translocation (FIG. 3), or μOR internalization (FIG. 4) and based upon these observations could be termed a non-desensitizing agonist. A non-desensitizing opioid might be promising in the development of analgesics that could activate the receptor while preventing receptor desensitization and ultimately avoid the development of antinociceptive tolerance.

The cellular trafficking of Compound Vb in vitro has been studied and correlated with behavioral responses in rats. A question remains as to whether the βarrestin translocation and internalization profiles observed in the cellular system are preserved at the biological sites of action in vivo. This is an important consideration as morphine has been shown to promote efficient internalization profiles in certain neuronal populations while promoting no internalization in other neurons (Keith, D. E., Murray, S. R., Zaki, P. A., Chu, P. C., Lissin, D. V., Kang, L., Evans, C. J. & von Zastrow, M., 1996, J Biol Chem 271, 19021-4; Haberstock-Debic, H., Kim, K. A., Yu, Y. J. & von Zastrow, M., 2005, J Neurosci 25, 7847-57; and Haberstock-Debic, H., Wein, M., Barrot, M., Colago, E. E., Rahman, Z., Neve, R. L., Pickel, V. M., Nestler, E. J., von Zastrow, M. & Svingos, A. L., 2003, J Neurosci 23, 4324-32). The HEK-293 cellular system has been used to assess the Compound Vb-induced μOR trafficking profiles since this model has been the most extensively used to study opioid receptor trafficking and should allow for the best overall comparison of Compound Vb to other opioids. Ultimately, receptor trafficking induced by Compound Vb should be evaluated in neurons that mediate its antinociceptive effects.

Compound Vb is biologically active in vivo as it is able to suppress pain responses in the rat formalin paw-withdrawal nociceptive assay in a naloxone reversible manner (FIG. 6A). Rats that received repeated doses of Compound Vb did not display a decreased response to the drug over time demonstrating that tolerance to the drug did not develop. Previous studies have shown that βarr2-KO mice do not develop morphine antinociceptive tolerance demonstrating the importance of the βarr2-μOR interaction in the development of opioid tolerance in vivo. The lack of Compound Vb-mediated βarrestin recruitment to the gμOR observed in vitro may be an underlying mechanism by which Compound Vb can continue to produce analgesia in the absence of tolerance in vivo. Future studies assessing βarrestin-μOR interactions in neurons should shed light on this mechanism.

Compound Vb was evaluated in a peripheral nociceptive test paradigm, the rat paw formalin test. It has previously been shown that opioids can mediate antinociceptive effects at receptors located on sensory nerves, including those within rat paw (Stein, C., Gramsch, C. & Herz, A., 1990, J Neurosci 10, 1292-8; and Stein, C., Gramsch, C., Hassan, A. H., Przewlocki, R., Parsons, C. G., Peter, K. & Herz, A., 1990, Prog Clin Biol Res 328, 425-7). Opioid actions at these receptors can promote antinociception either by directly inhibiting neuronal firing or by preventing the release of proinflammatory neuropeptides (Stein, C., 1995, N Engl J Med 332, 1685-90). The two phases of the formalin test have been associated with physiologically distinct pathways of nociception. The first phase is believed to reflect a direct activation of nociceptors while the second phase is due to an inflammatory response (Hunskaar, S. & Hole, K., 1987, Pain 30, 103-14). Opioids exert their antinociceptive effects on both phases, yet the second phase effects can be due to both peripheral and/or central nervous system sites of action. Since Compound Vb was injected directly into the site of the pain response and had suppressive effects that were most pronounced in the second phase of the test (FIG. 6C), it may be producing antinociception at peripheral opioid receptors.

Compound Vb retains some affinity at the κOR, therefore, nor-BNI was used to determine if its antinociceptive properties were due to activation of the θOR. Nor-BNI pretreatment did not prevent Compound Vb-induced antinociception, but rather, its analgesic properties were enhanced by the kappa antagonist pretreatment (FIG. 6A). Kappa-selective agonists have also been shown to be anti-analgesic in that their activation can oppose μOR-mediated analgesia (Pan, Z. Z., 1998, Trends Pharmacol Sci 19, 94-8).

Agonists that activate the μOR without the recruitment of βarrestins may have additional benefits besides the potential alleviation of antinociceptive tolerance. βarr2-KO mice, wherein receptors are activated in the absence of βarr2 regulation, display enhanced and prolonged morphine-induced antinociception (Bohn, L. M., Gainetdinov, R. R., Lin, F. T., Lefkowitz, R. J. & Caron, M. G., 2000, Nature 408, 720-3; Bohn, L. M., Lefkowitz, R. J. & Caron, M. G., 2002, J Neurosci 22, 10494-500; and Bohn, L. M., Belcheva, M. M. & Coscia, C. J., 2000, J Neurochem 74, 564-73). Recently, morphine-induced side effects, including constipation and respiratory suppression, were shown to be greatly diminished in mice lacking βarr2 (Raehal, K. M. & Bohn, L. M., 2005, Aaps J 7, E587-91). The mechanisms that determine how one μOR-mediated physiological response can be enhanced while another is suppressed due to the ablation of βarr2 is not known; however, these observations speak to the diverse roles that βarrestins may play in receptor signaling as well as regulation (Shenoy, S. K. & Lefkowitz, R. J., 2003, Biochem J 375, 503-15). If βarr2-opioid receptor interaction is required for opioid-induced constipation and respiratory suppression, then compounds that can activate the μOR and not recruit the βarrestins may be promising opioid analgesics with much fewer side effects.

### Example 24

The following illustrate representative pharmaceutical dosage forms, containing a compound of the invention (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/ml) | mg/ml |
| Compound X = (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (v) Injection 2 (10 mg/ml) | mg/ml |
| Compound X = (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |
| (vi) Aerosol | mg/can |
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 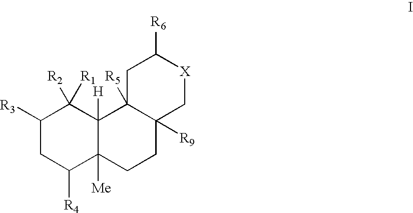
wherein:
R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;
R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;
R₄ is H, hydroxymethyl, (C₁-C₆)alkyl, (C₁-C₆)alkoxymethyl, carboxy, (C₁-C₆)alkoxycarbonyl or R_{d}RₑNC(═O)—;
R₅ is H or (C₁-C₆)alkyl;
R₆ is (C₁-C₆)alkyl, (C₁-C₆)cycloalkyl, aryl, Het, carboxy, RⱼRₖNC(═O)—, or heteroaryl;
R₉ is H or (C₁-C₆)alkyl;
X is —O—, —S—, or —NRₐ—;
each Rₐ is independently H, (C₁-C₆)alkyl, aryl, heteroaryl, aryl(C₁-C₆)alkyl, or heteroaryl(C₁-C₆)alkyl;
each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
each R_{c} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;
each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;
each R_{g} is independently (C₁-C₆)alkyl;
each Rₕ is independently H, (C₁-C₆)alkyl, fluoro, or chloro;
each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;
each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (Rₕ)₃C(═NR_{d})—, or R_{b}S(═O)₂—;
each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and
each R_{q} is independently H, (C₁-C₆)alkyl, (C₂-C₆)alkenyl, aryl, heteroaryl, aryl(C₁-C₆)alkyl, or heteroaryl(C₁-C₆)alkyl;
wherein any aryl or heteroaryl of R₃, R₆, and Rₐ-Rₑ, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂— or RᵤRᵥN; wherein Rᵤ and Rᵥ are each independently H or (C₁-C₆)alkyl;
each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and
wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;
or a pharmaceutically acceptable salt thereof,
provided the compound is not a compound of the following formula:

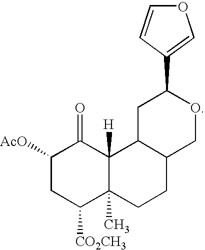

2. A compound of formula II: 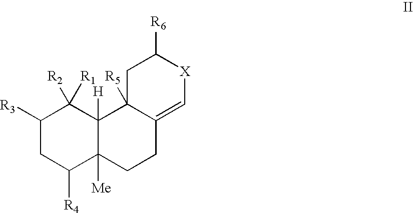
wherein:
R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;
R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;
R₄ is H, hydroxymethyl, (C₁-C₆)alkyl, (C₁-C₆)alkoxymethyl, carboxy, (C₁-C₆)alkoxycarbonyl or R_{d}RₑNC(═O)—;
R₅ is H or (C₁-C₆)alkyl;
R₆ is (C₁-C₆)alkyl, (C₁-C₆)cycloalkyl, aryl, Het, carboxy, RⱼRₖNC(═O)—, or heteroaryl;
X is —O—, —S—, or —NRₐ—;
each Rₐ is independently H, (C₁-C₆)alkyl, aryl, heteroaryl, aryl(C₁-C₆)alkyl, or heteroaryl(C₁-C₆)alkyl;
each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
each R_{c} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;
each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;
each R_{g} is independently (C₁-C₆)alkyl;
each Rₕ is independently H, (C₁-C₆)alkyl, fluoro, or chloro;
each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;
each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (Rₕ)₃C(═NR_{d})—, or R_{b}S(═O)₂—;
each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and
each R_{q} is independently H, (C₁-C₆)alkyl, (C₂-C₆)alkenyl, aryl, heteroaryl, aryl(C₁-C₆)alkyl, or heteroaryl(C₁-C₆)alkyl;
wherein any aryl or heteroaryl of R₃, R₆, and Rₐ-Rₑ and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;
each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and
wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ, and Rᵥ are each independently H or (C₁-C₆)alkyl;
or a pharmaceutically acceptable salt thereof;
provided the compound is not a compound of the following formula:

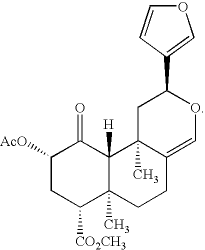

3. A compound of formula III: 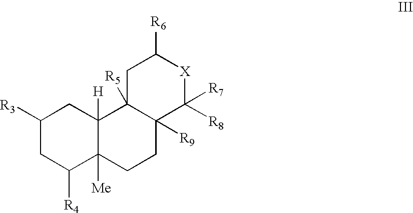
wherein:
R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;
R₄ is H, hydroxymethyl, (C₁-C₆)alkyl, (C₁-C₆)alkoxymethyl, carboxy, (C₁-C₆)alkoxycarbonyl or R_{d}RₑNC(═O)—;
R₅ is H or (C₁-C₆)alkyl;
R₆ is (C₁-C₆)alkyl, (C₁-C₆)cycloalkyl, aryl, Het, carboxy, RⱼRₖNC(═O)—, or heteroaryl;
R₇ and R₈ taken together are oxo (═O), thioxo (═S), or ═NRₐ;
R₉ is H or (C₁-C₆)alkyl;
X is —O—, —S—, or —NRₐ—;
each Rₐ is independently H, (C₁-C₆)alkyl, aryl, heteroaryl, aryl(C₁-C₆)alkyl, or heteroaryl(C₁-C₆)alkyl;
each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
each R_{c} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;
each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;
each R_{g} is independently (C₁-C₆)alkyl;
each Rₕ is independently H, (C₁-C₆)alkyl, fluoro, or chloro;
each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;
each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (Rₕ)₃C(═NR_{d})—, or R_{b}S(═O)₂—;
each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and
each R_{q} is independently H, (C₁-C₆)alkyl, (C₂-C₆)alkenyl, aryl, heteroaryl, aryl(C₁-C₆)alkyl, or heteroaryl(C₁-C₆)alkyl;
wherein any aryl or heteroaryl of R₃, R₆, and Rₐ-Rₑ, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂—, or RᵤRᵥN; wherein Rᵤ and Rᵥ are each independently H or (C₁-C₆)alkyl;
each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and
wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;
or a pharmaceutically acceptable salt thereof;
provided the compound is not a compound of the following formula: 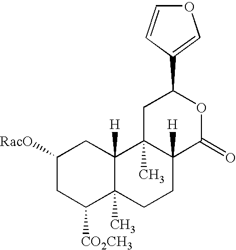
wherein R_{ac} is H or CH₃C(═O)—.

4. A compound of formula IV: 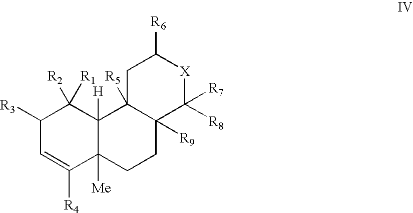
wherein:
R₁ is H, hydroxy, (C₁-C₆)alkyl, (C₁-C₆)alkoxy, or (C₁-C₆)alkanoyloxy and R₂ is H or (C₁-C₆)alkyl; or R₁ and R₂ taken together are oxo (═O), thioxo (═S), or ═NRₐ;
R₃ is H, halo, azido, hydroxy, (C₁-C₆)alkyl, (C₁-C₆)alkoxy, (C₁-C₆)alkylthio, (C₁-C₆)alkoxy(C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, formyloxy, acetoxy, R_{c}C(═O)O—, R_{c}C(═S)O—, R_{c}C(═O)S—, (R_{g})₃SiO—, R_{d}RₑNC(═O)O—, (Rₕ)₃C(═NR_{d})O—, RₘRₙN—, or R_{b}S(═O)₂O—;
R₄ is H, hydroxymethyl, (C₁-C₆)alkyl, (C₁-C₆)alkoxymethyl, carboxy, (C₁-C₆)alkoxycarbonyl or R_{d}RₑNC(═O)—;
R₅ is H or (C₁-C₆)alkyl;
R₆ is (C₁-C₆)alkyl, (C₁-C₆)cycloalkyl, aryl, Het, carboxy, RⱼRₖNC(═O)—, or heteroaryl;
R₇ and R₈ taken together are oxo (═O), thioxo (═S), or ═NRₐ;
R₉ is H or (C₁-C₆)alkyl;
X is —O—, —S—, or —NRₐ—;
each Rₐ is independently H, (C₁-C₆)alkyl, aryl, heteroaryl, aryl(C₁-C₆)alkyl, or heteroaryl(C₁-C₆)alkyl;
each R_{b} is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
each R_{c} is independently H, (C₂-C₆)alkyl, (C₂-C₆)alkenyl, (C₂-C₆)alkoxycarbonyl, aryl, heteroaryl, aryl(C₁-C₆)alkyl, Het, Het(C₁-C₆)alkyl, or heteroaryl(C₁-C₆)alkyl;
each R_{d} and Rₑ is independently H, (C₁-C₆)alkyl, (C₁-C₆)alkenyl, aryl, heteroaryl, aryl(C₁-C₆)alkyl, or heteroaryl(C₁-C₆)alkyl;
each R_{g} is independently (C₁-C₆)alkyl;
each Rₕ is independently H, (C₁-C₆)alkyl, fluoro, or chloro;
each Rⱼ and Rₖ is independently H, (C₁-C₆)alkyl, (C₁-C₆)alkenyl, aryl, heteroaryl, aryl(C₁-C₆)alkyl, Het, Het(C₁-C₆)alkyl, or heteroaryl(C₁-C₆)alkyl;
each Rₘ and Rₙ is independently H, (C₁-C₆)alkyl, (C₁-C₆)alkenyl, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, Het, Het(C₁-C₆)alkyl, Het(C₁-C₆)alkoxy, (C₁-C₆)alkanoyloxy, RₚC(═O)—, R_{d}RₑNC(═O)—, (Rₕ)₃C(═NR_{d})—, or R_{b}S(═O)₂—;
each Rₚ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl; and
each R_{q} is independently H, (C₁-C₆)alkyl, (C₂-C₆)alkenyl, aryl, heteroaryl, aryl(C₁-C₆)alkyl, or heteroaryl(C₁-C₆)alkyl;
wherein any aryl or heteroaryl of R₃, R₆, and Rₐ-Rₑ, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, RₜS(═O)₂— or RᵤRᵥN; wherein Rᵤ and Rᵥ are each independently H or (C₁-C₆)alkyl;
each Rₜ is independently H, (C₁-C₆)alkyl, (C₂-C₆)alkenyl, aryl, heteroaryl, aryl(C₁-C₆)alkyl, Het, Het(C₁-C₆)alkyl, or heteroaryl(C₁-C₆)alkyl;
wherein any aryl or heteroaryl of Rₜ is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RᵤRᵥN; wherein Rᵤ and Rᵥ are each independently H or (C₁-C₆)alkyl; and
wherein any Het of R₃, R₆, R_{b}, R_{c}, and Rⱼ-R_{q} is optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, oxo (═O), thioxo (═S), R_{q}S(═O)₂O—, aryl, heteroaryl, or RᵤRᵥN; wherein Rᵤ and Rᵥ are each independently H or (C₁-C₆)alkyl;
or a pharmaceutically acceptable salt thereof,
provided the compound is not a compound of the following formula: 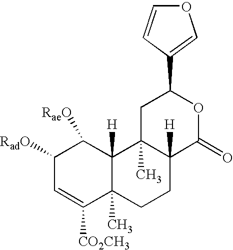
wherein R_{ad} is H or CH₃C(═O)—; and Rae is H or CH₃C(═O)—.

5. The compound of claim 1 wherein R₁ is hydroxy, (C₁-C₆)alkoxy, or (C₁-C₆)alkanoyloxy and R₂ is H.

6. The compound of claim 1 wherein R₁ and R₂ taken together are oxo (═O), thioxo (═S), or ═NRₐ.

7. The compound of claim 1 wherein R₁ and R₂ taken together are oxo (═O).

8. The compound of claim 1 wherein R₃ is H, hydroxy, (C₁-C₆)alkyl, (C₁-C₆)alkoxy, aryl, heteroaryl, aryloxy, heteroaryloxy, aryl(C₁-C₆)alkyl, aryl(C₁-C₆)alkoxy, heteroaryl(C₁-C₆)alkyl, heteroaryl(C₁-C₆)alkoxy, formyloxy, acetoxy, R_{c}C(═O)O—, (R_{g})₃SiO—, R_{d}RₑNC(═O)O—, (Rₕ)₃C(═NR_{d})O—, or R_{b}S(═O)₂O—.

9. The compound of claim 1 wherein R₃ is hydroxy, (C₁-C₆)alkoxy, aryloxy, heteroaryloxy, aryl(C₁-C₆)alkoxy, heteroaryl(C₁-C₆)alkoxy, formyloxy, R_{c}C(═O)O—, or R_{b}S(═O)₂O—.

10. The compound of claim 1 wherein R₃ is formyloxy, R_{c}C(═O)O—, or R_{b}S(═O)₂O—.

11. The compound of claim 1 wherein R₃ is propanoyloxy, isobutanoyloxy, methacryloyloxy, methoxyoxalyloxy, benzoyloxy, trimethylsilyloxy, imidazole-1-ylthiocarbonyloxy, methoxymethoxy, aminocarbonyloxy, butanoyloxy, pentanoyloxy, 1-bromobenzoyloxy, 2-bromobenzoyloxy, 3-bromobenzoyloxy, 4-methoxybenzoyloxy, 4-nitrobenzoyloxy, phenylsulfonyloxy, 4-methylphenylsulfonyloxy, 4-methoxyphenylsulfonyloxy, 4-bromophenylsulfonyloxy, (3-pyridylcarbonyloxy, methylsulfonyloxy, hydroxy, 1-imino-2,2,2-trichloroethoxy, phenylaminocarbonyloxy, alkylaminocarbonyloxy, 3,4-dichlorobenzoyloxy, bromo, azido, amino, acetylamino, phenylcarbonylamino, methylsulfonylamino, phenylsulfonylamino, or benzoyloxy.

12. The compound of claim 1 wherein R₃ propanoyloxy, methylsulfonyloxy, or benzoyloxy.

13. The compound of claim 1 wherein R₄ is hydroxymethyl, (C₁-C₆)alkoxymethyl, carboxy, (C₁-C₆)alkoxycarbonyl; or R_{d}RₑNC(═O)—.

14. The compound claim 1 wherein R₄ is carboxy, (C₁-C₆)alkoxycarbonyl; or R_{d}RₑNC(═O)—.

15. The compound of claim 1 wherein R₄ is methoxycarbonyl.

16. The compound of claim 1 wherein R₅ is H.

17. The compound of claim 1 wherein R₅ is methyl.

18. The compound of claim 1 wherein R₆ is aryl or heteroaryl, optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RₑR_{f}N.

19. The compound of claim 1 wherein R₆ is phenyl, thienyl, furanyl, pyrrolyl, or pyridyl, optionally substituted with one or more (e.g. 1, 2, 3, or 4) halo, hydroxy, (C₁-C₆)alkyl, (C₁-C₆)alkoxy, (C₁-C₆)alkanoyloxy, (C₁-C₆)alkoxycarbonyl, cyano, nitro, trifluromethyl, trifluoromethoxy, or RₑR_{f}N.

20. The compound of claim 1 wherein R₆ is 3-furyl.

21. The compound of claim 2 wherein R₇ and R₈ taken together are oxo.

22. The compound of claim 1 wherein X is —O—.

23. The compound of claim 1 wherein each Rₐ is independently H, methyl, ethyl, phenyl, thienyl, furanyl, pyrrolyl, pyridyl, benzyl, phenethyl, thienylmethyl, furanylmethyl, pyrrolylmethyl, or pyridylmethyl.

24. The compound of claim 1 wherein each R_{b} is independently H, (C₁-C₆)alkyl, (C₂-C₆)alkenyl, aryl, heteroaryl, aryl(C₁-C₆)alkyl, or heteroaryl(C₁-C₆)alkyl.

25. The compound of claim 1 wherein each R_{c} is independently H, ethyl, phenyl, thienyl, furanyl, pyrrolyl, pyridyl, benzyl, phenethyl, thienylmethyl, furanylmethyl, pyrrolylmethyl, or pyridylmethyl.

26. The compound of claim 1 wherein each R_{d} and Rₑ is independently H, methyl, ethyl, phenyl, thienyl, furanyl, pyrrolyl, pyridyl, benzyl, phenethyl, thienylmethyl, furanylmethyl, pyrrolylmethyl, or pyridylmethyl.

27. The compound of claim 1 which is a compound of formula (Ia): 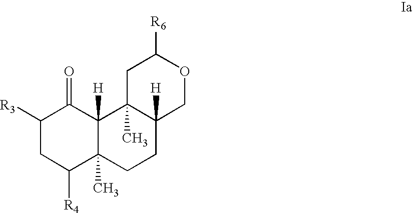
wherein R₃, R₄, and R₆ have any of the values defined in claim 1; or a pharmaceutically acceptable salt thereof.

28. The compound of claim 2 which is a compound of formula (IIa): 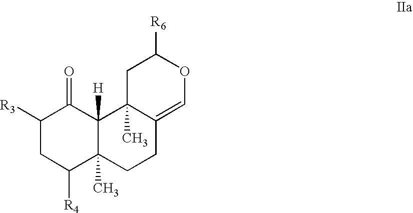
wherein R₃, R₄, and R₆ have any of the values defined in claim 1; or a pharmaceutically acceptable salt thereof.

29. The compound of claim 3 which is a compound of formula (IIIa): 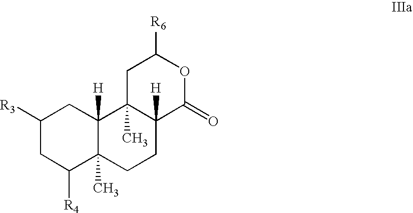
wherein R₃, R₄, and R₆ have any of the values defined in claim 1; or a pharmaceutically acceptable salt thereof.

30. The compound of claim 4 which is a compound of formula (IVa): 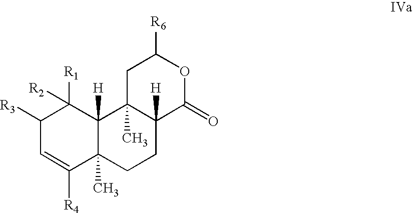
wherein R₁ is H, hydroxy, (C₁-C₆)alkoxy, or (C₁-C₆)alkanoyloxy and R₂ is H; and R₃, R₄, and R₆ have any of the values defined in claim 1; or a pharmaceutically acceptable salt thereof.

31-36. (canceled)

37. A pharmaceutical composition comprising a compound as described in claim 1 and a pharmaceutically acceptable diluent or carrier.

38. A method for modulating the activity of an opioid receptor comprising contacting the receptor with an effective modulatory amount of a compound as described in claim 1.

39-40. (canceled)

41. A therapeutic method for treating a disease or condition in an animal wherein the activity of an opioid receptor is implicated and modulation of the action of the receptor is desired comprising administering to the animal, an effective amount of a compound as described in claim 1.

42-71. (canceled)
